SEQUENCE LISTING (120 sequences)

SEQ ID NO: 1 - protein, 121 aa, Artificial Sequence
  Heavy Chain Variable region
QVQLVQSGSELKKPGASVKVSCKASGYTFTNFAMNWVRRAPGQGLEWMGWINTNTGNPTYAQGFTGRFVFSLDTSVNTAYLQISSLKAEDTAVYYCARDWGVIGGHYMDVWGKGTTVTVSS

SEQ ID NO: 2 - protein, 5 aa, Artificial Sequence
  HCDR1 according to KABAT  from SEQ ID NO 1
NFAMN

SEQ ID NO: 3 - protein, 17 aa, Artificial Sequence
  HCDR2 according to KABAT  from SEQ ID NO 1
WINTNTGNPTYAQGFTG

SEQ ID NO: 4 - protein, 12 aa, Artificial Sequence
  HCDR3 according to KABAT  from SEQ ID NO 1
DWGVIGGHYMDV

SEQ ID NO: 5 - protein, 121 aa, Artificial Sequence
  Heavy Chain Variable region
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSDGYGPKAFDYWGQGTLVTVSS

SEQ ID NO: 6 - protein, 5 aa, Artificial Sequence
  HCDR1 according to KABAT  from SEQ ID NO 5
SYGIS

SEQ ID NO: 7 - protein, 17 aa, Artificial Sequence
  HCDR2 according to KABAT  from SEQ ID NO 5
WISAYNGNTNYAQKLQG

SEQ ID NO: 8 - protein, 12 aa, Artificial Sequence
  HCDR3 according to KABAT  from SEQ ID NO 5
DSDGYGPKAFDY

SEQ ID NO: 9 - protein, 123 aa, Artificial Sequence
  Heavy Chain Variable region
EVQLVQSGAEVKKPGESLKISCKGSGYSFTSYWIGWVRQMPGKGLEWMGIIYPDDSDTRYSPSFQGQVTISADKSSSTAYLQWSSLKASDTAMYYCASFYTGIVGATGAFDVWGQGTTVTVSS

SEQ ID NO: 10 - protein, 5 aa, Artificial Sequence
  HCDR1 according to KABAT  from SEQ ID NO 9
SYWIG

SEQ ID NO: 11 - protein, 17 aa, Artificial Sequence
  HCDR2 according to KABAT  from SEQ ID NO 9
IIYPDDSDTRYSPSFQG

SEQ ID NO: 12 - protein, 14 aa, Artificial Sequence
  HCDR3 according to KABAT  from SEQ ID NO 9
FYTGIVGATGAFDV

SEQ ID NO: 13 - protein, 128 aa, Artificial Sequence
  Heavy Chain Variable region
QVQLVQSGAEVKKPGSSVKVSCKASGGTFSSDAISWVRQAPGQGLEWMGGMIPILGTANYAQKFQGRVTITADRSTSTAYMELSSLRSEDTAVYYCVRGATYYYGSGTYYSINWFDPWGQGTLVTVSS

SEQ ID NO: 14 - protein, 5 aa, Artificial Sequence
  HCDR1 according to KABAT  from SEQ ID NO 13
SDAIS

SEQ ID NO: 15 - protein, 17 aa, Artificial Sequence
  HCDR2 according to KABAT  from SEQ ID NO 13
GMIPILGTANYAQKFQG

SEQ ID NO: 16 - protein, 19 aa, Artificial Sequence
  HCDR3 according to KABAT  from SEQ ID NO 13
GATYYYGSGTYYSINWFDP

SEQ ID NO: 17 - protein, 122 aa, Artificial Sequence
  Heavy Chain Variable region
QVQLVQSGSELKKPGASVKVSCRASGYTFTNFAMTWVRQAPGQGPEYMGWINTNTGNPTYAQGFTGRFVFSLDTSVNTAYLQISSLKAEDTAVYYCARDWASVMVRGDLDYWGQGTLVTVSS

SEQ ID NO: 18 - protein, 5 aa, Artificial Sequence
  HCDR1 according to KABAT  from SEQ ID NO 17
NFAMT

SEQ ID NO: 19 - protein, 13 aa, Artificial Sequence
  HCDR3 according to KABAT  from SEQ ID NO 17
DWASVMVRGDLDY

SEQ ID NO: 20 - protein, 126 aa, Artificial Sequence
  Heavy Chain Variable region
QVQLVQSGAEVKKPGASVKVSCKVSGYTLSELSIHWVRQAPGKGVEWMGGFYPEDVEPIYARKFQGRVTMTEDTSTDTAYMELNSLRSEDTAVYYCAAEGFDNYGSGIRGNWFDPWGQGTLVTVSS

SEQ ID NO: 21 - protein, 5 aa, Artificial Sequence
  HCDR1 according to KABAT  from SEQ ID NO 20
ELSIH

SEQ ID NO: 22 - protein, 17 aa, Artificial Sequence
  HCDR2 according to KABAT  from SEQ ID NO 20
GFYPEDVEPIYARKFQG

SEQ ID NO: 23 - protein, 17 aa, Artificial Sequence
  HCDR3 according to KABAT  from SEQ ID NO 20
EGFDNYGSGIRGNWFDP

SEQ ID NO: 24 - protein, 122 aa, Artificial Sequence
  Heavy Chain Variable region
EVQLVQSGAEVKKPGASVKVSCKVSGYTLTELSMHWVRQSPGKGLEWMGSFYPEDGETIYAQKFQGRITMTEDTSADTAYMELSSLRSEDTAVYYCATEGVGVIRGNWFDPWGQGTLVTVSS

SEQ ID NO: 25 - protein, 5 aa, Artificial Sequence
  HCDR1 according to KABAT  from SEQ ID NO 24
ELSMH

SEQ ID NO: 26 - protein, 17 aa, Artificial Sequence
  HCDR2 according to KABAT  from SEQ ID NO 24
SFYPEDGETIYAQKFQG

SEQ ID NO: 27 - protein, 13 aa, Artificial Sequence
  HCDR3 according to KABAT  from SEQ ID NO 24
EGVGVIRGNWFDP

SEQ ID NO: 28 - protein, 123 aa, Artificial Sequence
  Heavy Chain Variable region
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSS

SEQ ID NO: 29 - protein, 17 aa, Artificial Sequence
  HCDR2 according to KABAT  from SEQ ID NO 28
IIFPDDSDTRYSPSFQG

SEQ ID NO: 30 - protein, 14 aa, Artificial Sequence
  HCDR3 according to KABAT  from SEQ ID NO 28
LGGYSGYAEDFVDF

SEQ ID NO: 31 - protein, 124 aa, Artificial Sequence
  Heavy Chain Variable region
EVQLVQSGAEVKKPGASVKVSCKVSGYTLTKLSMHWVRQAPGKGLEWMGGFEPEDGETINAQKFQGRVTMTEDTSTDTAYMELSSLRSEDTAVYYCATDLRLGASYYYSYMDVWGRGTMVTVSS

SEQ ID NO: 32 - protein, 5 aa, Artificial Sequence
  HCDR1 according to KABAT  from SEQ ID NO 31
KLSMH

SEQ ID NO: 33 - protein, 17 aa, Artificial Sequence
  HCDR2 according to KABAT  from SEQ ID NO 31
GFEPEDGETINAQKFQG

SEQ ID NO: 34 - protein, 15 aa, Artificial Sequence
  HCDR3 according to KABAT  from SEQ ID NO 31
DLRLGASYYYSYMDV

SEQ ID NO: 35 - protein, 121 aa, Artificial Sequence
  Heavy Chain Variable region
QITLKESGPTLVKPTQTLTLSCTFSGFSLSTSGMSVGWIRQPPGKALEWLALIYWNDDKYFSPSLKSRLTITKDTSKNQVVLTLTNMDPVDTATYYCAHTLWGSDDVFDVWGQGTMVTVSS

SEQ ID NO: 36 - protein, 7 aa, Artificial Sequence
  HCDR1 according to KABAT  from SEQ ID NO 35
TSGMSVG

SEQ ID NO: 37 - protein, 16 aa, Artificial Sequence
  HCDR2 according to KABAT  from SEQ ID NO 35
LIYWNDDKYFSPSLKS

SEQ ID NO: 38 - protein, 11 aa, Artificial Sequence
  HCDR3 according to KABAT  from SEQ ID NO 35
TLWGSDDVFDV

SEQ ID NO: 39 - protein, 123 aa, Artificial Sequence
  Heavy Chain Variable region
EVQLVQSGAEVKKPGESLKISCKVSGYSFTNYWIGWVRQMPGKGLEWMGIIYPGDSDTRYSPSFQGQVTISADKSISTAYLQWHTLKASDTAMYYCARHQGYSFSGSHIDDYWGQGTLVTVSS

SEQ ID NO: 40 - protein, 5 aa, Artificial Sequence
  HCDR1 according to KABAT  from SEQ ID NO 39
NYWIG

SEQ ID NO: 41 - protein, 17 aa, Artificial Sequence
  HCDR2 according to KABAT  from SEQ ID NO 39
IIYPGDSDTRYSPSFQG

SEQ ID NO: 42 - protein, 14 aa, Artificial Sequence
  HCDR3 according to KABAT  from SEQ ID NO 39
HQGYSFSGSHIDDY

SEQ ID NO: 43 - protein, 123 aa, Artificial Sequence
  Heavy Chain Variable region
EVQLVQSGAEVRKPGESLKISCKGSGYSFTTYWIGWVRQMPGKGLEWMGIIYPGDSDTRYSPSFQGQVTISADKSISTVYLQWSSLKASDTAMYYCARHAGFIITSQNIDDYWGQGTLVTVSS

SEQ ID NO: 44 - protein, 5 aa, Artificial Sequence
  HCDR1 according to KABAT  from SEQ ID NO 43
TYWIG

SEQ ID NO: 45 - protein, 14 aa, Artificial Sequence
  HCDR3 according to KABAT  from SEQ ID NO 43
HAGFIITSQNIDDY

SEQ ID NO: 46 - protein, 121 aa, Artificial Sequence
  Heavy Chain Variable region
EVQLVQSGSELKKPGASVKVSCKASGYTFTNFAMNWVRQAPGQGLEWMGWINTNTGNPTYAQDFTGRFVFSLDTSGNTAYLQISSLKAEDTAVYYCARDWGLVAIGYFDYWGQGTLVTVSS

SEQ ID NO: 47 - protein, 17 aa, Artificial Sequence
  HCDR2 according to KABAT  from SEQ ID NO 46
WINTNTGNPTYAQDFTG

SEQ ID NO: 48 - protein, 12 aa, Artificial Sequence
  HCDR3 according to KABAT  from SEQ ID NO 46
DWGLVAIGYFDY

SEQ ID NO: 49 - protein, 124 aa, Artificial Sequence
  Heavy Chain Variable region
QITLKESGPTLVKPTQTLTLTCTFSGFSLSTTGVGVNWIRQPPGEALEWLALIYWNDDTYYSPSLKSRLTITKDTSKNQVVLTMTNMDPVDTATYYCAHEGIIGFLGGNWFDPWGQGTLVTVSS

SEQ ID NO: 50 - protein, 7 aa, Artificial Sequence
  HCDR1 according to KABAT  from SEQ ID NO 49
TTGVGVN

SEQ ID NO: 51 - protein, 16 aa, Artificial Sequence
  HCDR2 according to KABAT  from SEQ ID NO 49
LIYWNDDTYYSPSLKS

SEQ ID NO: 52 - protein, 14 aa, Artificial Sequence
  HCDR3 according to KABAT  from SEQ ID NO 49
EGIIGFLGGNWFDP

SEQ ID NO: 53 - protein, 125 aa, Artificial Sequence
  Heavy Chain Variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 54 - protein, 5 aa, Artificial Sequence
  HCDR1 according to KABAT  from SEQ ID NO 53
SHAMN

SEQ ID NO: 55 - protein, 17 aa, Artificial Sequence
  HCDR2 according to KABAT  from SEQ ID NO 53
WINPNTGNPTYAQGFTG

SEQ ID NO: 56 - protein, 16 aa, Artificial Sequence
  HCDR3 according to KABAT  from SEQ ID NO 53
DRKYVTNWVFAEDFQH

SEQ ID NO: 57 - protein, 125 aa, Artificial Sequence
  Heavy Chain Variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCAIDRGYMSNWVFAEYFPHWGQGTLVTVSS

SEQ ID NO: 58 - protein, 16 aa, Artificial Sequence
  HCDR3 according to KABAT  from SEQ ID NO 57
DRGYMSNWVFAEYFPH

SEQ ID NO: 59 - protein, 125 aa, Artificial Sequence
  Heavy Chain Variable region
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCATDRGYISSWVFAEDFQHWGQGTLVTVSS

SEQ ID NO: 60 - protein, 5 aa, Artificial Sequence
  HCDR1 according to KABAT  from SEQ ID NO 59
SYAMN

SEQ ID NO: 61 - protein, 16 aa, Artificial Sequence
  HCDR3 according to KABAT  from SEQ ID NO 59
DRGYISSWVFAEDFQH

SEQ ID NO: 62 - protein, 125 aa, Artificial Sequence
  Heavy Chain Variable region
QVQLVQSGSELKKPGASVKVSCTASGYTFTSYAMNWVRQAPGQRLEWMACVNPNTGSPTYAQGSTGRFVVSLDTSVSTAYLQISSLKAEDTAVYYCARDRKYVTNWVFAEDFQHWGHGTLVTVSS

SEQ ID NO: 63 - protein, 17 aa, Artificial Sequence
  HCDR2 according to KABAT  from SEQ ID NO 62
CVNPNTGSPTYAQGSTG

SEQ ID NO: 64 - protein, 125 aa, Artificial Sequence
  Heavy Chain Variable region
QVQLVQSGSELKKPGASVKVSCKASGYTFTNYAMNWVRQAPGQGLEWMGWMNPNTGNPTYAQGSTGRFVVSLDTSVSTAYLQISSLKAEDTAVYYCARDRKYVTNWVFAEDFQHWGRGTLVTVSS

SEQ ID NO: 65 - protein, 5 aa, Artificial Sequence
  HCDR1 according to KABAT  from SEQ ID NO 64
NYAMN

SEQ ID NO: 66 - protein, 17 aa, Artificial Sequence
  HCDR2 according to KABAT  from SEQ ID NO 64
WMNPNTGNPTYAQGSTG

SEQ ID NO: 67 - protein, 125 aa, Artificial Sequence
  Heavy Chain Variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGRGTLVTVSS

SEQ ID NO: 68 - protein, 5 aa, Artificial Sequence
  HCDR1 according to KABAT  from SEQ ID NO 67
NYAIN

SEQ ID NO: 69 - protein, 124 aa, Artificial Sequence
  Heavy Chain Variable region
EVQLVQSGAEVKKPGSSVKVSCKASGDTFNTYSITWVRQAPGQGLEWMGSIVPIFGTINNAQKFQGRVTITADKSANTAYMELSSLRSEDTAVYYCARDNTMVRGVDYYYMDVWGKGTMVTVSS

SEQ ID NO: 70 - protein, 5 aa, Artificial Sequence
  HCDR1 according to KABAT  from SEQ ID NO 69
TYSIT

SEQ ID NO: 71 - protein, 17 aa, Artificial Sequence
  HCDR2 according to KABAT  from SEQ ID NO 69
SIVPIFGTINNAQKFQG

SEQ ID NO: 72 - protein, 15 aa, Artificial Sequence
  HCDR3 according to KABAT  from SEQ ID NO 69
DNTMVRGVDYYYMDV

SEQ ID NO: 73 - protein, 121 aa, Artificial Sequence
  Heavy Chain Variable region
EVQLVQSGAEVKKPGSSVKVSCKASGGIFSTYAISWVRQAPGQGLEWMGGIIPIFDTPNYAQKFQGRVTITADKSTSTAYMDLSSLRSEDTAVYYCAKNVRGYSAYDLDYWGQGTLVTVSS

SEQ ID NO: 74 - protein, 5 aa, Artificial Sequence
  HCDR1 according to KABAT  from SEQ ID NO 73
TYAIS

SEQ ID NO: 75 - protein, 17 aa, Artificial Sequence
  HCDR2 according to KABAT  from SEQ ID NO 73
GIIPIFDTPNYAQKFQG

SEQ ID NO: 76 - protein, 12 aa, Artificial Sequence
  HCDR3 according to KABAT  from SEQ ID NO 73
NVRGYSAYDLDY

SEQ ID NO: 77 - protein, 119 aa, Artificial Sequence
  Heavy Chain Variable region
EVQLVQSGAEVKNPGSSVKVSCKATGGTFNTYGTNWVRQAPGQGLEWMGGIIPIFGTANYAQKFQGRVTITADKSTTTAYMEVSSLRSEDTAVYYCARGGADMGTLDYWGQGTLVTVSS

SEQ ID NO: 78 - protein, 5 aa, Artificial Sequence
  HCDR1 according to KABAT  from SEQ ID NO 77
TYGTN

SEQ ID NO: 79 - protein, 17 aa, Artificial Sequence
  HCDR2 according to KABAT  from SEQ ID NO 77
GIIPIFGTANYAQKFQG

SEQ ID NO: 80 - protein, 10 aa, Artificial Sequence
  HCDR3 according to KABAT  from SEQ ID NO 77
GGADMGTLDY

SEQ ID NO: 81 - protein, 120 aa, Artificial Sequence
  Heavy Chain Variable region
EVQLVQSGAEVMRPGSSVKVSCKASGGIFNTYTIIWVRQAPGQGLEWMGGIIPIFDTPNFAQKFQGRLTITADKSTNTAYMELTSLRSEDTAVYYCAREGCNHGVCYPYWGQGTLVTVSS

SEQ ID NO: 82 - protein, 5 aa, Artificial Sequence
  HCDR1 according to KABAT  from SEQ ID NO 81
TYTII

SEQ ID NO: 83 - protein, 17 aa, Artificial Sequence
  HCDR2 according to KABAT  from SEQ ID NO 81
GIIPIFDTPNFAQKFQG

SEQ ID NO: 84 - protein, 11 aa, Artificial Sequence
  HCDR3 according to KABAT  from SEQ ID NO 81
EGCNHGVCYPY

SEQ ID NO: 85 - protein, 121 aa, Artificial Sequence
  Heavy Chain Variable region
QVQLVQSGAEVKKPGSSVKVSCKASGDTFRSYGITWVRQAPGQGLEWMGGIIPIFGTTNYAQKFQGRVTITADKSTSTVYMELSSLRSEDTAVYYCARRRGYSNPHWLDPWGQGTLVTVSS

SEQ ID NO: 86 - protein, 5 aa, Artificial Sequence
  HCDR1 according to KABAT  from SEQ ID NO 85
SYGIT

SEQ ID NO: 87 - protein, 17 aa, Artificial Sequence
  HCDR2 according to KABAT  from SEQ ID NO 85
GIIPIFGTTNYAQKFQG

SEQ ID NO: 88 - protein, 12 aa, Artificial Sequence
  HCDR3 according to KABAT  from SEQ ID NO 85
RRGYSNPHWLDP

SEQ ID NO: 89 - protein, 119 aa, Artificial Sequence
  Heavy Chain Variable region
QVQLVQSGAEVKKPGSSVKVSCKASGGTFSTYGILWVRQAPGQGLEWMGGIIPIFGTANYAQKFQGRVTITADISTSTAYMELSSLRSEDTAVYYCARGGGNYYEFVYWGQGTLVTVSS

SEQ ID NO: 90 - protein, 5 aa, Artificial Sequence
  HCDR1 according to KABAT  from SEQ ID NO 89
TYGIL

SEQ ID NO: 91 - protein, 10 aa, Artificial Sequence
  HCDR3 according to KABAT  from SEQ ID NO 89
GGGNYYEFVY

SEQ ID NO: 92 - protein, 121 aa, Artificial Sequence
  Heavy Chain Variable region
EVQLVQSGAEVKKPGSSVRVSCKASGGTFNTYAINWVRQAPGQGLEWVGRIIPIFDTANYAQKFQGRVTISADKSTTTAYMELSSLRSEDTAVFYCAKDETGYSSSNFQHWGQGTLVTVSS

SEQ ID NO: 93 - protein, 5 aa, Artificial Sequence
  HCDR1 according to KABAT  from SEQ ID NO 92
TYAIN

SEQ ID NO: 94 - protein, 17 aa, Artificial Sequence
  HCDR2 according to KABAT  from SEQ ID NO 92
RIIPIFDTANYAQKFQG

SEQ ID NO: 95 - protein, 12 aa, Artificial Sequence
  HCDR3 according to KABAT  from SEQ ID NO 92
DETGYSSSNFQH

SEQ ID NO: 96 - protein, 125 aa, Artificial Sequence
  Heavy Chain Variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 97 - protein, 122 aa, Artificial Sequence
  Heavy Chain Variable region
QVQLVQSGAEVKRPGSSVKVSCKASGGTFNTYSITWVRQAPGQGLEWMGGIIPVFGTSKYAQKFQDRVTITADKSTNTAYMELSSLRSEDTAVYYCARDPSFSSSSGWFDPWGQGTLVTVSS

SEQ ID NO: 98 - protein, 17 aa, Artificial Sequence
  HCDR2 according to KABAT  from SEQ ID NO 97
GIIPVFGTSKYAQKFQD

SEQ ID NO: 99 - protein, 13 aa, Artificial Sequence
  HCDR3 according to KABAT  from SEQ ID NO 97
DPSFSSSSGWFDP

SEQ ID NO: 100 - protein, 121 aa, Artificial Sequence
  HEAVY CHAIN VARIABLE REGION
QVQLVQSGAEVKKPGSSVKVSCKASGGTFNTYAINWVRQAPGQGLEWMGGIIPIFDTANYAQRFQGRVTITADKSTSTAYMELSSLRSEDTAVYFCAKDQTGYSSTLFDYWGQGTLVTVSS

SEQ ID NO: 101 - protein, 17 aa, Artificial Sequence
  HCDR2 according to KABAT  from SEQ ID NO 100
GIIPIFDTANYAQRFQG

SEQ ID NO: 102 - protein, 12 aa, Artificial Sequence
  HCDR3 according to KABAT  from SEQ ID NO 100
DQTGYSSTLFDY

SEQ ID NO: 103 - protein, 125 aa, Artificial Sequence; identical to SEQ ID NO: 57
  Heavy Chain Variable region

SEQ ID NO: 104 - protein, 121 aa, Artificial Sequence
  Heavy Chain Variable region
EVQLVQSGAEVKKPGSSVKVSCKASGGTFSTYAISWVRQAPGQGLEWMGWIIPIFDTGNYAQKIQGRVTITADKSTSTAYMELTSLRSEDTAVYYCARHDYTNTVDAFDIWGQGTMVTVSS

SEQ ID NO: 105 - protein, 17 aa, Artificial Sequence
  HCDR2 according to KABAT  from SEQ ID NO 104
WIIPIFDTGNYAQKIQG

SEQ ID NO: 106 - protein, 12 aa, Artificial Sequence
  HCDR3 according to KABAT  from SEQ ID NO 104
HDYTNTVDAFDI

SEQ ID NO: 107 - protein, 121 aa, Artificial Sequence
  Heavy Chain Variable region
QVQLVQSGAEVKKPGSSVKVSCKASGDTFRSYGITWVRQAPGQGLEWMGGIIPVFGTTNYAQKFQGRVTITADKSTSTVFMELNSLRSEDTAVYYCARRRGYSNPHWLDPWGQGTLVTVSS

SEQ ID NO: 108 - protein, 17 aa, Artificial Sequence
  HCDR2 according to KABAT  from SEQ ID NO 107
GIIPVFGTTNYAQKFQG

SEQ ID NO: 109 - protein, 214 aa, Artificial Sequence
  Amino acid sequence of human common light chain IGKV1-39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 107 aa, Artificial Sequence
  Amino acid sequence of common light chain variable domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 111 - protein, 11 aa, Artificial Sequence
  LCDR1 according to KABAT  from SEQ ID NO 110
RASQSISSYLN

SEQ ID NO: 112 - protein, 7 aa, Artificial Sequence
  LCDR2 according to KABAT  from SEQ ID NO 110
AASSLQS

SEQ ID NO: 113 - protein, 9 aa, Artificial Sequence
  LCDR3 according to KABAT  from SEQ ID NO 110
QQSYSTPPT

SEQ ID NO: 114 - protein, 107 aa, Artificial Sequence [sequence omitted]

SEQ ID NO: 115 - protein, 107 aa, Artificial Sequence [sequence omitted]

SEQ ID NO: 116 - protein, 98 aa, Artificial Sequence
  Amino acid sequence of CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 117 - protein, 15 aa, Artificial Sequence
  Amino acid sequence of the hinge
EPKSCDKTHTCPPCP

SEQ ID NO: 118 - protein, 110 aa, Artificial Sequence
  Amino acid sequence of CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 119 - protein, 107 aa, Artificial Sequence
  Amino acid sequence of CH3 with KK mutations
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 107 aa, Artificial Sequence
  Amino acid sequence of CH3 with DE mutations
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0118277 A1
(43) Pub. Date: Apr. 11, 2024

(54) METHOD FOR DETECTING EXPRESSION OR CLUSTERING OF CELL SURFACE MOIETIES

(71) Applicant: MERUS N.V., Utrecht (NL)

(72) Inventor: Cecilia Anna Wilhelmina GEUIJEN, Utrecht (NL)

(21) Appl. No.: 18/190,607

(22) PCT Filed: Sep. 28, 2021
(86) PCT No.: PCT/EP2021/076688

§ 371 Date: Mar. 27, 2023

(30) Foreign Application Priority Data
Sep. 28, 2020 (NL) 2026558

(51) Int. Cl. G01N 33/566; G01N 33/574

(52) U.S. Cl. CPC G01N 33/566; G01N 33/57492; G01N 2500/02; G01N 2800/52

(57) ABSTRACT

The present disclosure relates to a method for detecting and/or quantifying expression of at least a first cell surface moiety and of a second cell surface moiety in a patient sample, and to a method for detecting and/or quantifying clustering of at least a first cell surface moiety with a second cell surface moiety in a sample, wherein the sample is exposed to a molecule having binding specificity for the at least first and second cell surface moieties. The present disclosure further relates to a method for predicting the responsiveness of a subject to such binding molecule, a method for determining the effectiveness of such binding molecule, a method for confirming the mode of action of such binding molecule, a method for treating a subject, and a method for screening one or more test agents for the ability to induce clustering of a first cell surface moiety with a second cell surface moiety.

### FIELD

The present disclosure relates to a method for detecting and/or quantifying expression, or the clustering, of at least a first cell surface moiety and of a second cell surface moiety, preferably in a patient tumor sample. in certain embodiments, the methods of the present disclosure can be used to predict if a patient is likely to benefit from therapy with a binding agent that binds both cell surface moieties, or to confirm the mode of action of such binding agent.

### BACKGROUND

The development of therapeutic antibodies for the treatment of cancer has rapidly increased over the past years. For a variety of cancer types, diagnostic assays are being used to assess whether a certain patient will benefit from treatment with a particular drug, such that it can be predicted that it is likely to be safe and/or effective. One category of diagnostic assays which has been used with biologics or large molecule therapeutics involves the testing of expression levels of an antigen targeted by a biologic in a patient's tissue sample. For example, a tissue biopsy can be taken from a patient's tumor and subjected to a quantitative assay. Examples of such quantitative assays include immunohistochemistry (IHC), dual in situ hybridization assay, chromogenic in situ hybridization (CISH) assay, and fluorescent in situ hybridization (FISH) assay.

IHC has been a standard testing method for the evaluation of for instance HER2 expression, for example, in breast cancer. IHC testing utilizes specific monoclonal or polyclonal antibodies which bind to the HER2 protein on the cellular surface. The addition of a secondary tagged antibody having a reporter function, followed by an enzymatic reaction, yields a signal that is proportional to the amount of HER2 protein present. However, despite guidelines on the grading and scoring of IHC expression levels inter-laboratory variability cannot be completely prevented.

FISH, CISH, and silver-enhanced in situ hybridization assays quantify the gene copy number per cell using a single- or dual-probe technique. FISH has become a widely accepted platform in, for instance HER2, testing. However, FISH assays are costly, labor-intensive, and require fluorescent microscopy and advanced training. Bright-field in situ hybridization assays such as CISH and silver-enhanced in situ hybridization do not require fluorescent microscopy and are less costly.

Another development is an assay that has been validated as a method to measure for instance total HER2, HER2 homodimers, or p95HER2 expression in breast cancer, which is a proximity-based assay designed to quantify protein expression, dimerization, and protein-protein interaction (described in detail in Diagn Mol Pathol 2009; 18:11-21; Shi et al.).

To date, the art has lacked a robust method of demonstrating the capacity of two or more antigens to cluster in the presence of a multispecific agent, where such antigens do not normally associate under somatic conditions. Here, the inventors demonstrate such an assay, and exemplary applications of its uses.

### SUMMARY

The present inventors have developed an assay for detecting and quantifying the expression levels of CD137, a cell surface moiety expressed on T cells, and of PD-L1, a cell surface moiety expressed on tumor cells. This allows for the prediction of whether a particular patient is likely to respond to, and benefit from, a treatment binding these two cell surface moieties. In certain embodiments, other applicable methods may be used that, like the assay used herein, are based on measuring a signal that is produced when the two cell surface moieties are present in the same sample. In this context, a signal can be the presence or absence of a signal. In certain embodiments, both such read-outs provide information about the expression levels of the cell surface moieties. Also, the method may be used to detect and/or quantify the expression levels of any two or more cell surface moieties that can be bound by a particular drug, such as for instance a multispecific agent, like a bispecific or trispecific, antibody.

The present inventors have further developed an assay used for detecting and quantifying the clustering of CD137, and of CD137 with PD-L1. CD137 and PD-L1 do not form a cognate receptor-ligand pair, they do not naturally cluster or form a direct protein-protein interaction. However, when targeted by a drug that simultaneously binds to both cell surface moieties, such as for instance a multispecific agent, like a bispecific or trispecific antibody, these two cell surface moieties are brought in proximity of each other thereby producing a signal that can be detected. Similarly, when targeted by a multivalent drug that binds to at least two of the same cell surface moieties, such as for instance a monospecific, bivalent agent, like a monospecific antibody, or a multispecific agent, like a bispecific or trispecific antibody, the at least two cell surface moieties are brought in proximity of each other thereby producing a signal that can be detected.

In certain embodiments, the present disclosure is based on the therapeutic use of a multispecific agent, such as a bi- or trispecific antibody, that simultaneously binds to two or more target antigens on the cell surface of tumor cells and/or cells from the immune system. The multispecific agent therewith induces the clustering of the two or more target antigens. The clustering of the two or more antigens thus only occurs in the presence of a multispecific agent, or occurs at an increased level compared to when no multispecific agent is present.

In certain embodiments, this allows for the assessment of whether the drug that a patient is being treated with is indeed binding the two cell surface moieties simultaneously and exhibits its expected mode of action. In certain embodiments, one may use other applicable methods that, like the assay used herein, are based on measuring a signal that is produced when the two cell surface moieties are in close proximity. In this context, a signal can be the presence or absence of a reporter or feature of the assay. In certain embodiments, both such read-outs (presence or loss of a reporter) provide information about the proximity of the cell surface moieties. Also, the method may be used to detect and/or quantify the clustering of any two or more cell surface moieties that can simultaneously be bound by a particular drug, such as for instance a multispecific agent, like a bispecific or trispecific antibody.

In certain embodiments, the present disclosure relates to a method for detecting and/or quantifying the presence in a sample of clustering of at least two cell surface moieties, comprising a first cell surface moiety and a second cell surface moiety, the method comprising:
contacting a sample in which the first and second cell surface moieties have been exposed to an agent having binding specificity for at least the first and second cell surface moieties with a first binding molecule that specifically binds to the first cell surface moiety and a second binding molecule that specifically binds to the second cell surface moiety, wherein at least one of the first binding molecule and the second binding molecule comprises a molecular tag which is not detected unless the first and second cell surface moieties are in proximity of each other; and
detecting the presence or absence of the molecular tag to detect the presence in the sample of clustering of the first cell surface moiety and the second cell surface moiety.

The present disclosure also relates to a method for detecting and/or quantifying expression in a sample of at least two cell surface moieties, comprising a first cell surface moiety and a second cell surface moiety, the method comprising:
contacting a tumor biopsy sample from a subject having cancer with at least one binding molecule that detects a first cell surface moiety and at least one binding molecule that detects a second cell surface moiety,
wherein at least one binding molecule that detects the first cell surface moiety and at least one binding molecule that detects the second cell surface moiety comprise a molecular tag;
and
detecting and/or quantifying the presence or absence of the molecular tags to detect the expression of the first cell surface moiety and of the second cell surface moiety in the sample.

The present disclosure further relates to a method for predicting the responsiveness of a subject, in particular a cancer patient, to an agent or agents binding a first cell surface moiety and a second cell surface moiety, in particular a moiety expressed on an immune effector cell and a moiety expressed on a tumor cell, the method comprising:
detecting and/or quantifying the expression levels of a first cell surface moiety and a second cell surface moiety in a biological sample from a subject;
determining whether the expression levels of the first cell surface moiety and the second cell surface moiety in the subject's sample is above or below a threshold level; and
predicting that the subject is likely to respond to an agent or agents binding the first cell surface moiety and the second cell surface moiety if the expression levels of the first cell surface moiety and the second cell surface moiety in the subject's sample is equal to or above the threshold level.

The present disclosure further relates to a method for treating a subject in need thereof, having cancer, the method comprising:
predicting responsiveness of a subject, in particular a subject having cancer, to an agent or agents binding a first cell surface moiety and a second cell surface moiety as described herein; and
administering an agent or agents binding the first cell surface moiety and the second cell surface moiety to a subject that is likely to respond.

The present disclosure further relates to a method for determining the effectiveness of an agent, the agent comprising a binding molecule that comprises at least a binding domain that specifically binds to a first cell surface moiety and a binding domain that specifically binds to a second cell surface moiety, the method comprising detecting and/or quantifying clustering of a first cell surface moiety with a second cell surface moiety in a biological sample of a subject under treatment with the agent, as described herein.

The present disclosure further relates to a method for confirming the mode of action of an agent, the agent comprising a binding molecule that comprises at least a binding domain that specifically binds to a first cell surface moiety and a binding domain that specifically binds to a second cell surface moiety, the method comprising detecting and/or quantifying clustering of a first cell surface moiety with a second cell surface moiety in a biological sample of a subject under treatment with the agent, as described herein.

The present disclosure further relates to a method for treating a subject in need thereof, in particular a subject having cancer, the method comprising:
treating a subject in need thereof with an agent binding a first cell surface moiety and a second cell surface moiety;
analyzing the effectiveness of the agent or the mode of action of the agent, as described herein.

The present disclosure further relates to a method for screening one or more test agents for the ability to induce clustering of a first cell surface moiety with a second cell surface moiety, the method comprising:
contacting one or more test cell cultures with a test agent,
wherein the test cell culture comprises a cell expressing a first cell surface moiety, and a cell expressing a second cell surface moiety;
detecting the level of clustering of the first and second cell surface moieties, as described herein; and
comparing the level of clustering with the level of clustering detected for the clustering in a control cell culture not contacted with the test agent or contacted with a reference agent,
wherein the control cell culture comprises the first cell surface moiety, and the second cell surface moiety.

### DESCRIPTION OF THE DRAWINGS

FIG. 1. Schematic representation of the concept of a VeraTag® assay used according to certain embodiments of the present disclosure. The VeraTag® assay format in this representation makes use of two primary antibodies that bind one or two particular antigens, one of the primary antibodies comprising a tag and the other a cleavage-inducing moiety (scissors symbol). Photoactivation releases the cleavage-inducing moiety from one of the antibodies, where upon the cleavage-inducing moiety induces cleavage of the tag from the other antibody. The signal produced by the tag is subsequently measured by capillary electrophoresis (CE).

The disclosures set out herein are, however, not limited to the assay format presented in this figure.

FIG. 2. Schematic representation of an example of a format of a VeraTag® assay using primary antibodies. In this representation, the first and second cell surface moieties are different cell surface moieties. The present disclosure, however, also includes embodiments where the first and second cell surface moieties are the same.

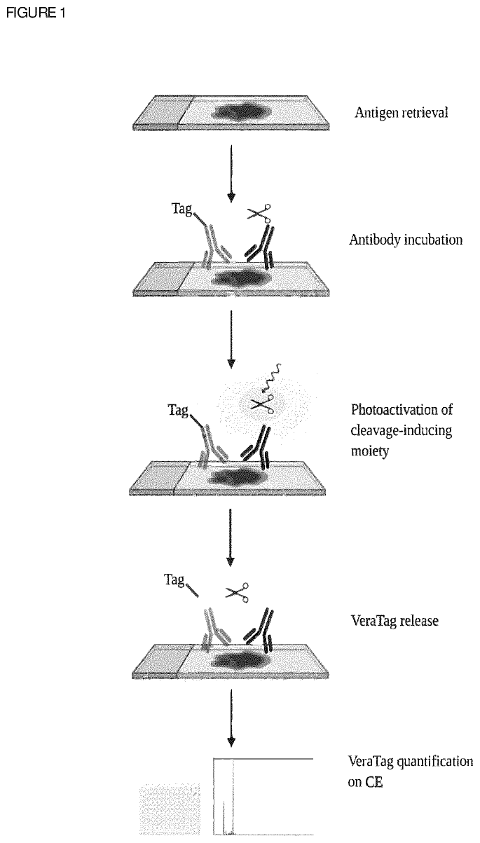

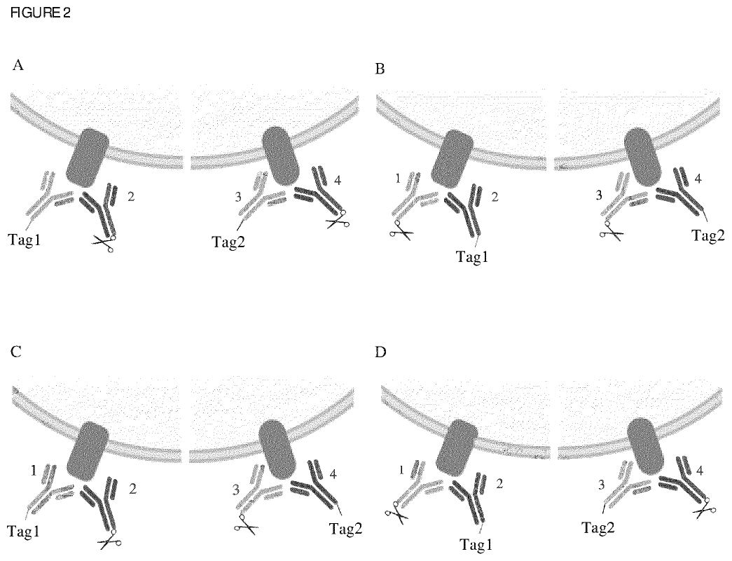

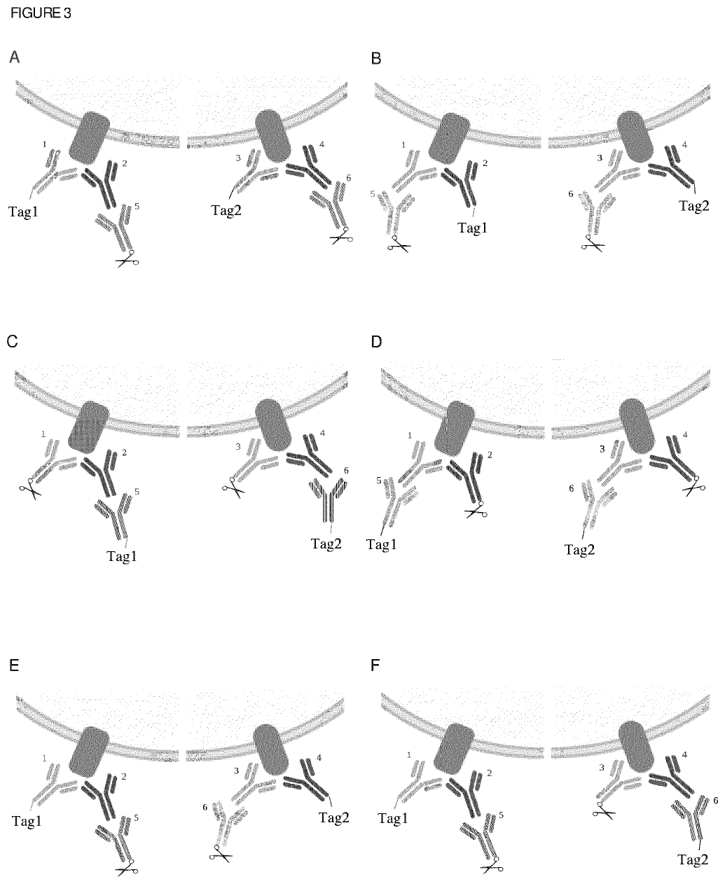

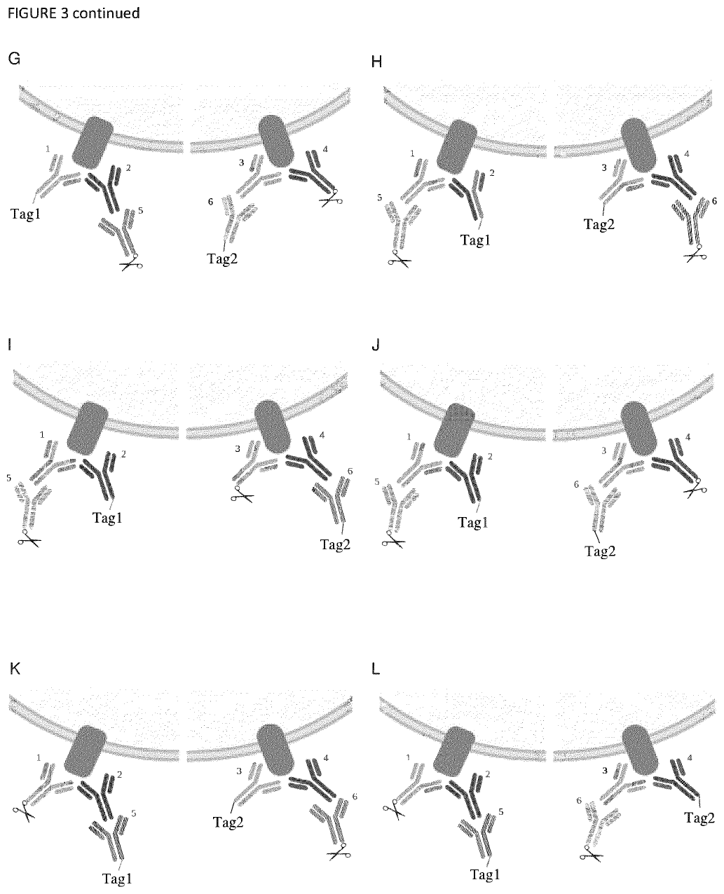

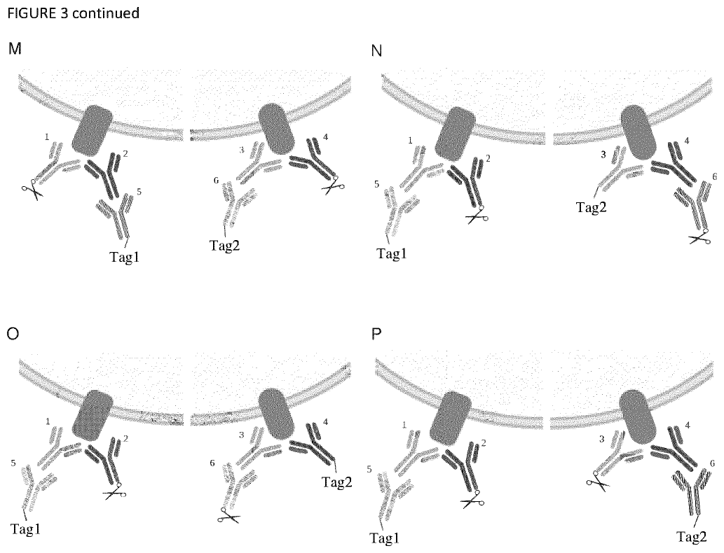

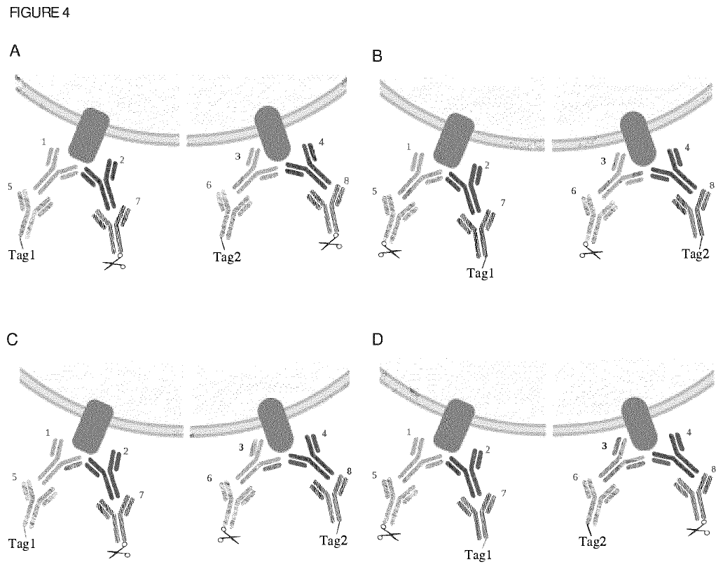

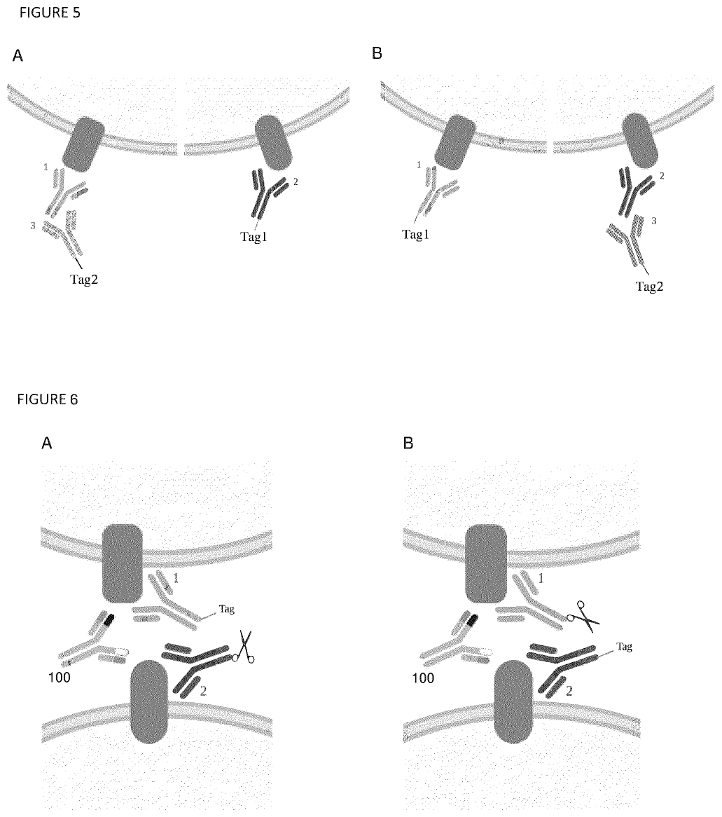

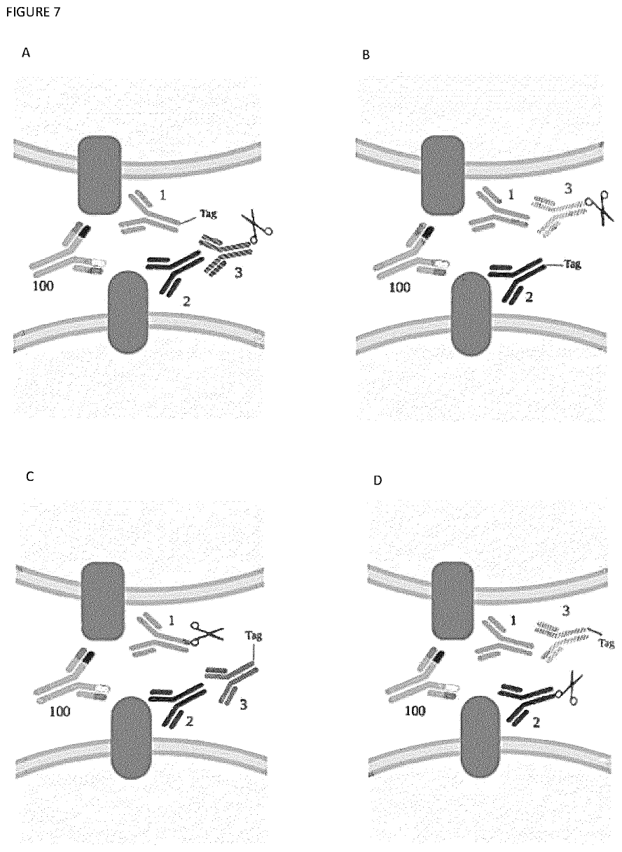

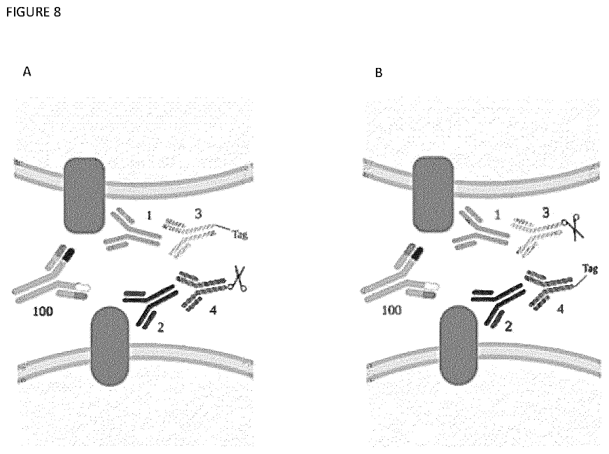

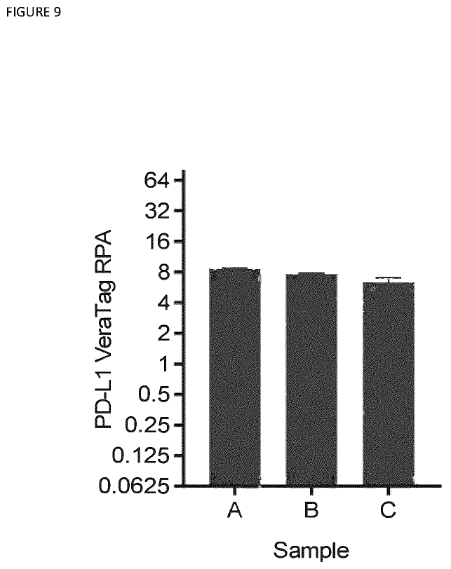

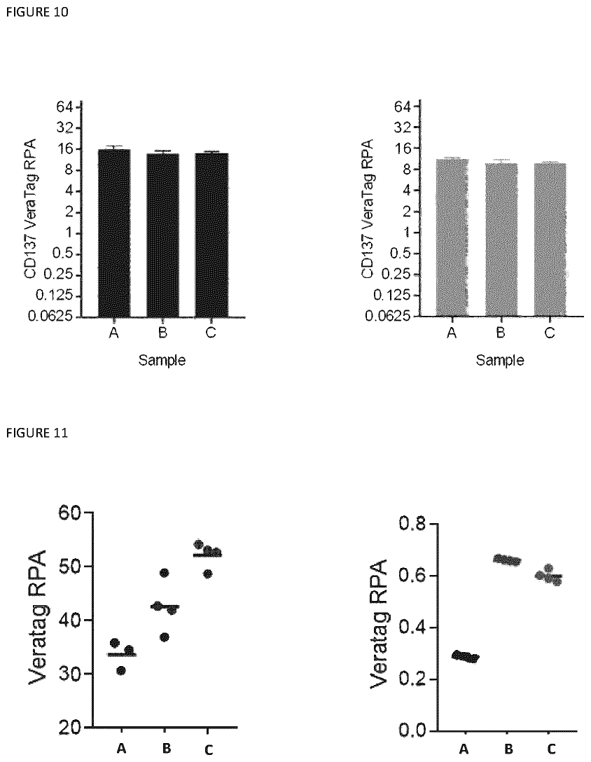

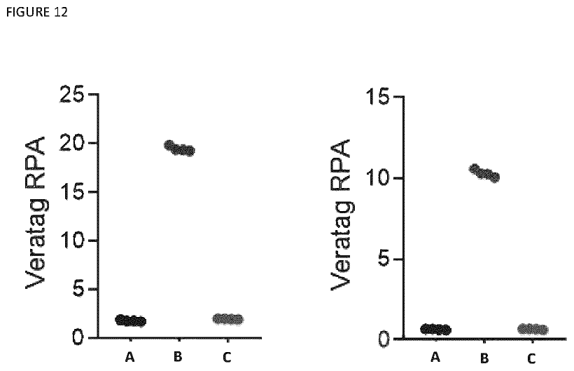

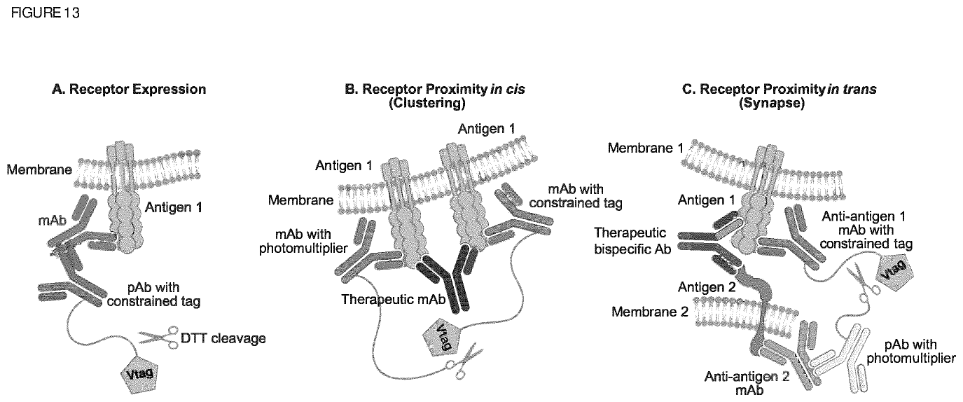

A) a first and second binding molecule that specifically bind to a first moiety expressed on a cell surface and a third and fourth binding molecule that specifically bind to a second moiety expressed on a cell surface, wherein the first binding molecule comprises a first molecular tag attached thereto via a cleavable linker and the second binding molecule comprises a cleavage inducing moiety (scissors symbol), and wherein the third binding molecule comprises a second molecular tag attached thereto via a cleavable linker and the fourth binding molecule comprises a cleavage inducing moiety (scissors symbol);

B) a first and second binding molecule that specifically bind to a first moiety expressed on a cell surface and a third and fourth binding molecule that specifically bind to a second moiety expressed on a cell surface, wherein the second binding molecule comprises a first molecular tag attached thereto via a cleavable linker and the first binding molecule comprises a cleavage inducing moiety (scissors symbol), and wherein the fourth binding molecule comprises a second molecular tag attached thereto via a cleavable linker and the third binding molecule comprises a cleavage inducing moiety (scissors symbol);

C) a first and second binding molecule that specifically bind to a first moiety expressed on a cell surface and a third and fourth binding molecule that specifically bind to a second moiety expressed on a cell surface, wherein the first binding molecule comprises a first molecular tag attached thereto via a cleavable linker and the second binding molecule comprises a cleavage inducing moiety (scissors symbol), and wherein the fourth binding molecule comprises a second molecular tag attached thereto via a cleavable linker and the third binding molecule comprises a cleavage inducing moiety (scissors symbol);

D) a first and second binding molecule that specifically bind to a first moiety expressed on a cell surface and a third and fourth binding molecule that specifically bind to a second moiety expressed on a cell surface, wherein the second binding molecule comprises a first molecular tag attached thereto via a cleavable linker and the first binding molecule comprises a cleavage inducing moiety (scissors symbol), and wherein the third binding molecule comprises a second molecular tag attached thereto via a cleavable linker and the fourth binding molecule comprises a cleavage inducing moiety (scissors symbol).

FIG. 3. Schematic representation of an example of a format of a VeraTag® assay using two primary antibodies and a secondary antibody for each target. In this representation, the first and second cell surface moieties are different cell surface moieties. The present disclosure, however, also includes embodiments where the first and second cell surface moieties are the same.

A) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first and third binding molecules comprise a first and second molecular tag respectively, attached thereto via a cleavable linker, the fifth binding molecule binds to the second binding molecule and comprises a cleavage inducing moiety (scissors symbol), and the sixth binding molecule binds to the fourth binding molecule and comprises a cleavage inducing moiety (scissors symbol);

B) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the second and fourth binding molecules comprise a first and second molecular tag respectively, attached thereto via a cleavable linker, the fifth binding molecule binds to the first binding molecule and comprises a cleavage inducing moiety (scissors symbol), and the sixth binding molecule binds to the third binding molecule and comprises a cleavage inducing moiety (scissors symbol);

C) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first and third binding molecules comprise a cleavage inducing moiety (scissors symbol), the fifth binding molecule binds to the second binding molecule and comprises a first molecular tag attached thereto via a cleavable linker, and the sixth binding molecule binds to the fourth binding molecule and comprises a second molecular tag attached thereto via a cleavable linker;

D) a first and second binding molecule that specifically bind to a first moiety expressed on a on a cell surface, a fifth binding molecule, and a sixth binding molecule,

wherein the second and fourth binding molecules comprise a cleavage inducing moiety (scissors symbol), the fifth binding molecule binds to the first binding molecule and comprises a first molecular tag attached thereto via a cleavable linker, and the sixth binding molecule binds to the third binding molecule and comprises a second molecular tag attached thereto via a cleavable linker;

E) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first binding molecule comprises a first molecular tag attached thereto via a cleavable linker, the fourth binding molecule comprises a second molecular tag attached thereto via a cleavable linker, the fifth binding molecule binds to the second binding molecule and comprises a cleavage inducing moiety (scissors symbol), and the sixth binding molecule binds to the third binding molecule and comprises a cleavage inducing moiety (scissors symbol);

F) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first binding molecule comprises a first molecular tag attached thereto via a cleavable linker, the third binding molecule comprises a cleavage inducing moiety (scissors symbol), the fifth binding molecule binds to the second binding molecule and comprises a cleavage inducing moiety (scissors symbol), and the sixth binding molecule binds to the fourth binding molecule and comprises a second molecular tag attached thereto via a cleavable linker;

G) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first binding molecule comprises a first molecular tag attached thereto via a cleavable linker, the fourth binding molecule comprises a cleavage inducing moiety (scissors symbol), the fifth binding molecule binds to the second binding molecule and comprises a cleavage inducing moiety (scissors symbol), and the sixth binding molecule binds to the third binding molecule and comprises a second molecular tag attached thereto via a cleavable linker;

H) a first and second binding molecule that specifically bind to a first moiety expressed on a on a cell surface, a fifth binding molecule, and a sixth binding molecule,

wherein the second binding molecule comprises a first molecular tag attached thereto via a cleavable linker, the third binding molecules comprises a second molecular tag attached thereto via a cleavable linker, the fifth binding molecule binds to the first binding molecule and comprises a cleavage inducing moiety (scissors symbol), and the sixth binding molecule binds to the fourth binding molecule and comprises a cleavage inducing moiety (scissors symbol);

I) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the second binding molecule comprises a first molecular tag attached thereto via a cleavable linker, the third binding molecule comprises a cleavage inducing moiety (scissors symbol), the fifth binding molecule binds to the first binding molecule and comprises a cleavage inducing moiety (scissors symbol), and the sixth binding molecule binds to the fourth binding molecule and comprises second molecular tag attached thereto via a cleavable linker;

J) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the second binding molecule comprises a first molecular tag attached thereto via a cleavable linker, the fourth binding molecule comprises a cleavage inducing moiety (scissors symbol), the fifth binding molecule binds to the first binding molecule and comprises a cleavage inducing moiety (scissors symbol), and the sixth binding molecule binds to the third binding molecule and comprises a second molecular tag attached thereto via a cleavable linker;

K) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first binding molecule comprises a cleavage inducing moiety (scissors symbol), the third binding molecule comprises a second molecular tag attached thereto via a cleavable linker, the fifth binding molecule binds to the second binding molecule and comprises a first molecular tag attached thereto via a cleavable linker, and the sixth binding molecule binds to the fourth binding molecule and comprises a cleavage inducing moiety (scissors symbol);

L) a first and second binding molecule that specifically bind to a first moiety expressed on a on a cell surface, a fifth binding molecule, and a sixth binding molecule,

wherein the first binding molecule comprises a cleavage inducing moiety (scissors symbol), the fourth binding molecule comprises a second molecular tag attached thereto via a cleavable linker, the fifth binding molecule binds to the second binding molecule and comprises a first molecular tag attached thereto via a cleavable linker, and the sixth binding molecule binds to the third binding molecule and comprises a cleavage inducing moiety (scissors symbol);

M) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the first binding and fourth binding molecules comprise a cleavage inducing moiety (scissors symbol), the fifth binding molecule binds to the second binding molecule and comprises a first molecular tag attached thereto via a cleavable linker, and the sixth binding molecule binds to the third binding molecule and comprises a second molecular tag attached thereto via a cleavable linker;

N) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the second binding molecule comprise a cleavage inducing moiety (scissors symbol), the third binding molecule comprises a second molecular tag attached thereto via a cleavable linker, the fifth binding molecule binds to the first binding molecule and comprises a first molecular tag attached thereto via a cleavable linker, and the sixth binding molecule binds to the fourth binding molecule and comprises a cleavage inducing moiety (scissors symbol);

O) a first and second binding molecule that specifically bind to a first moiety expressed on a cell surface, a third and fourth binding molecule that specifically bind to a second moiety expressed on a cell surface, a fifth binding molecule, and a sixth binding molecule,

wherein the second binding molecule comprises a cleavage inducing moiety (scissors symbol), the fourth binding molecule comprises a second molecular tag attached thereto via a cleavable linker, the fifth binding molecule binds to the first binding molecule and comprises a first molecular tag attached thereto via a cleavable linker, and the sixth binding molecule binds to the third binding molecule and comprises a cleavage inducing moiety (scissors symbol);

P) a first and second binding molecule that specifically bind to a first moiety expressed on a on a cell surface, a fifth binding molecule, and a sixth binding molecule,

wherein the second and third binding molecules comprise a cleavage inducing moiety (scissors symbol), the fifth binding molecule binds to the first binding molecule and comprises a first molecular tag attached thereto via a cleavable linker, and the sixth binding molecule binds to the fourth binding molecule and comprises a second molecular tag attached thereto via a cleavable linker.

FIG. 4. Schematic representation of an example of a format of a VeraTag® assay using two primary antibodies and two secondary antibodies for each target. In this representation, the first and second cell surface moieties are different cell surface moieties. The present disclosure, however, also includes embodiments where the first and second cell surface moieties are the same.

A) a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,

wherein the fifth binding molecule binds to the first binding molecule and comprises a first molecular tag attached thereto via a cleavable linker, the seventh binding molecule binds to the second binding molecule and comprises a cleavage inducing moiety (scissors symbol), the sixth binding molecule binds to the third binding molecule and comprises a second molecular tag attached thereto via a cleavable linker, and the eighth binding molecule binds to the fourth binding molecule and comprises a cleavage inducing moiety (scissors symbol);

B) a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,

wherein the fifth binding molecule binds to the first binding molecule and comprises a cleavage inducing moiety (scissors symbol), the seventh binding molecule binds to the second binding molecule and comprises a first molecular tag attached thereto via a cleavable linker, the sixth binding molecule binds to the third binding molecule and comprises a cleavage inducing moiety (scissors symbol), and the eighth binding molecule binds to the fourth binding molecule and comprises a second molecular tag attached thereto via a cleavable linker;

C) a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,

wherein the fifth binding molecule binds to the first binding molecule and comprises a first molecular tag attached thereto via a cleavable linker, the seventh binding molecule binds to the second binding molecule and comprises a cleavage inducing moiety (scissors symbol), the sixth binding molecule binds to the third binding molecule and comprises a cleavage inducing moiety (scissors symbol), and the eighth binding molecule binds to the fourth binding molecule and comprises a second molecular tag attached thereto via a cleavable linker;

D) a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,

wherein the fifth binding molecule binds to the first binding molecule and comprises a cleavage inducing moiety (scissors symbol), the seventh binding molecule binds to the second binding molecule and comprises a first molecular tag attached thereto via a cleavable linker, the sixth binding molecule binds to the third binding molecule and comprises a second molecular tag attached thereto via a cleavable linker, and the eighth binding molecule binds to the fourth binding molecule and comprises a cleavage inducing moiety (scissors symbol).

FIG. 5. Schematic representation of an example of a format of a VeraTag® assay using one primary antibody for each target and one secondary antibody against one of the primary antibodies. This format uses DTT-mediated release of the molecular tag. In this representation, the first and second cell surface moieties are different cell surface moieties. The present disclosure, however, also includes embodiments where the first and second cell surface moieties are the same.

A) a first binding molecule that specifically binds to a first moiety expressed on a cell surface, a second binding molecule that specifically binds to a second moiety expressed on a cell surface, and a third binding molecule that binds to the first binding molecule,

wherein the second binding molecule comprises a first molecular tag attached thereto via a cleavable linker; and wherein the third binding molecule comprises a second molecular tag attached thereto via a cleavable linker;

B) a first binding molecule that specifically binds to a first moiety expressed on a cell surface, a second binding molecule that specifically binds to a second moiety expressed on a cell surface, and a third binding molecule that binds to the second binding molecule,

wherein the first binding molecule comprises a first molecular tag attached thereto via a cleavable linker; and wherein the third binding molecule comprises a second molecular tag attached thereto via a cleavable linker.

FIG. 6. Schematic representation of an example of a format of a VeraTag® assay using one primary antibody for each target, for detecting and/or quantifying clustering of the targets. In this representation, the first and second cell surface moieties are different cell surface moieties expressed on different cells. The present disclosure, however, also includes embodiments where the first and second cell surface moieties are present on the same cell. Also, in this representation the molecule having binding specificity for the first and second cell surface moieties is a bivalent bispecific antibody. However, the present disclosure also encompasses the situation wherein the molecule having binding specificity for the cell surface moieties is a multivalent bispecific antibody or a multispecific, such as a trispecific or tetraspecific, antibody.

A) a bispecific antibody (100) binding a first cell surface moiety and a second cell surface moiety, thereby bringing the first and second cell surface moieties in proximity of each other; a first binding molecule that specifically binds to the first cell surface moiety and a second binding molecule that specifically binds to the second cell surface moiety,

wherein the first binding molecule comprises a molecular tag attached thereto via a cleavable linker and the second binding molecule comprises a cleavage inducing moiety (scissors symbol);

B) a bispecific antibody (100) binding a first cell surface moiety and a second cell surface moiety, thereby bringing the first and second cell surface moieties in proximity of each other; a first binding molecule that specifically binds to the first cell surface moiety and a second binding molecule that specifically binds to the second cell surface moiety,

wherein the second binding molecule comprises a molecular tag attached thereto via a cleavable linker and the first binding molecule comprises a cleavage inducing moiety.

FIG. 7. Schematic representation of an example of a format of a VeraTag® assay using one primary antibody for each target and a secondary antibody against one of the primary antibodies, for detecting and/or quantifying clustering of the targets. In this representation, the first and second cell surface moieties are different cell surface moieties expressed on different cells. The present disclosure, however, also includes embodiments where the first and second cell surface moieties are present on the same cell. Also, in this representation the molecule having binding specificity for the first and second cell surface moieties is a bivalent bispecific antibody. However, the present disclosure also encompasses the situation wherein the molecule having binding specificity for the cell surface moieties is a multivalent bispecific antibody or a multispecific, such as a trispecific or tetraspecific, antibody.

A) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, and a third binding molecule, wherein the first binding molecule comprises a molecular tag attached thereto via a cleavable linker and the third binding molecule binds to the second binding molecule and comprises a cleavage inducing moiety (scissor symbol);

B) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, and a third binding molecule, wherein the second binding molecule comprises a molecular tag attached thereto via a cleavable linker and the third binding molecule binds to the first binding molecule and comprises a cleavage inducing moiety (scissors symbol);

C) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, and a third binding molecule, wherein the first binding molecule comprises a cleavage inducing moiety (scissors symbol) and the third binding molecule binds to the second binding molecule and comprises a molecular tag attached thereto via a cleavable linker;

D) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, and a third binding molecule, wherein the second binding molecule comprises a cleavage inducing moiety (scissors symbol) and the third binding molecule binds to the first binding molecule and comprises a molecular tag attached thereto via a cleavable linker.

FIG. 8. Schematic representation of a format of a VeraTag® assay using one primary antibody for each target and two secondary antibodies against the primary antibodies, for detecting and/or quantifying clustering of the targets. In this representation, the first and second cell surface moieties are different cell surface moieties expressed on different cells. The present disclosure, however, also extends to where the first and second cell surface moieties are present on the same cell. Also, in this representation the molecule having binding specificity for the first and second cell surface moieties is a bivalent bispecific antibody. However, the present disclosure also encompasses the situation wherein the molecule having binding specificity for the cell surface moieties is a multivalent bispecific antibody or a multispecific, such as a trispecific or tetraspecific, antibody.

A) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, a third binding molecule, and a fourth binding molecule, wherein the third binding molecule binds to the first binding molecule and comprises a molecular tag attached thereto via a cleavable linker, and the fourth binding molecule binds to the second binding molecule and comprises a cleavage inducing moiety (scissors symbol);

B) a bispecific antibody (100) binding a first cell surface moiety and a second cell surface moiety, thereby bringing the first and second cell surface moieties in proximity of each other; a first binding molecule that specifically binds to the first cell surface moiety, a second binding molecule that specifically binds to the second cell surface moiety, a third binding molecule, and a fourth binding molecule, wherein the third binding molecule binds to the first binding molecule and comprises a cleavage inducing moiety (scissors symbol); and the fourth binding molecule binds to the second binding molecule and comprises a molecular tag attached thereto via a cleavable linker.

FIG. 9. Diagram showing PD-L1 expression levels in Relative Peak Area (RPA), measured using a VeraTag® assay. Sample A: cell pellet prepared by incubation with an anti-CD137 positive control antibody; sample B: cell pellet prepared by incubation with a bispecific antibody binding to CD137 and PD-L1; sample C: cell pellet prepared by incubation with a negative control antibody binding to RSV.

FIG. 10. Diagrams showing CD137 expression levels in Relative Peak Area (RPA), measured using a VeraTag® assay. Left: incubation with anti-CD137 assay antibody BBK2; right: incubation with anti-CD137 assay antibody M127. Sample A: cell pellet prepared by incubation with an anti-CD137 positive control antibody; sample B: cell pellet prepared by incubation with a bispecific antibody binding to CD137 and PD-L1; sample C: cell pellet prepared by incubation with a negative control antibody binding to RSV.

FIG. 11. Graphs showing CD137 clustering in Relative Peak Area (RPA), measured using a VeraTag® assay. Left: incubation with anti-CD137 assay antibody BBK2; right: incubation with anti-CD137 assay antibody M127. Sample A: cell pellet prepared by incubation with a negative control antibody binding to RSV; sample B: cell pellet prepared by incubation with a bispecific antibody binding to CD137 and PD-L1; sample C: cell pellet prepared by incubation with an anti-CD137 positive control antibody.

FIG. 12. Diagrams showing PD-L1-CD137 clustering in Relative Peak Area (RPA), measured using a VeraTag® assay. Left: incubation with anti-CD137 assay antibody BBK2; right: incubation with anti-CD137 assay antibody M127. Sample A: cell pellet prepared by incubation with a negative control antibody binding to RSV; sample B: cell pellet prepared by incubation with a bispecific antibody binding to CD137 and PD-L1; sample C: cell pellet prepared by incubation with an anti-CD137 positive control antibody. The level of clustering in this assay is compared to that measured in an isotype control experiment (ITC).

FIG. 13. Schematic representation of: A—an assay as described herein for detecting expression of a receptor (Antigen 1) present on a cell membrane; B—a proximity assay as described herein for detecting clustering a receptor (Antigen 1) on the same cell; and C—a proximity assay as described herein for detecting clustering of a receptor (Antigen 1) on one cell with a receptor (Antigen 2) on another cell, thereby forming an immunological synapse.

### DETAILED DESCRIPTION

In one aspect, the present disclosure provides a method for detecting and/or quantifying expression of at least a first cell surface moiety and of a second cell surface moiety in a sample, the method comprising detecting a signal produced when the first and second cell surface moieties are expressed in the same sample. In certain embodiments, preferably two different signals are produced so that it is possible to quantify the expression of each cell surface moiety. Alternatively, in certain embodiments, the signal-producing moieties are selected such that a combined signal provides information about the expression level of each cell surface moiety.

A signal can be produced in a variety of ways, including but not limited to, providing the first and second cell surface moieties with, preferably different, fluorescent molecular tags, providing the first and second cell surface moieties with, preferably different, chromogenic molecular tags, providing the first and second cell surface moieties with, preferably different, radioactive molecular tags; and providing the first and second cell surface moieties with, preferably different, isotopically pure metal chelator molecular tags. Quenching is another method that can be used. In certain embodiments, preferably different fluorophores are used for each cell surface moiety to permit the measurement of a reduction in intensity of fluorescence of one fluorophore or signal from a signal emitting agent caused by the interaction with a second quencher.

The method according to the present disclosure can be performed in different formats. In one format, the present disclosure provides a method for detecting and/or quantifying expression in a sample of at least two cell surface moieties, comprising a first cell surface moiety and a second cell surface moiety, the method comprising:
a) contacting a sample with at least one binding molecule that detects a first cell surface moiety and at least one binding molecule that detects a second cell surface moiety,
wherein at least one binding molecule that detects the first cell surface moiety and at least one binding molecule that detects the second cell surface moiety comprise a molecular tag, optionally wherein the molecular tag attached to the binding molecule that detects the first cell surface moiety is different from the molecular tag attached to the binding molecule that detects the second cell surface moiety; and
b) detecting the presence or absence, or measuring the amount, of the molecular tags to detect and/or quantify the expression of the first cell surface moiety and of the second cell surface moiety in the sample.

In another format, the present disclosure provides a method for detecting and/or quantifying expression in a sample of at least two cell surface moieties, comprising a first cell surface moiety and a second cell surface moiety, the method comprising:
a) contacting a sample with at least two binding molecules that detect a first cell surface moiety and at least two binding molecules that detect a second cell surface moiety,
wherein one of the binding molecules that detects the first cell surface moiety and one of the binding molecules that detects the second cell surface moiety comprise a molecular tag, preferably attached thereto via a cleavable linker, and optionally wherein another of the binding molecules that detects the first cell surface moiety and another of the binding molecules that detects the second cell surface moiety comprise a cleavage inducing moiety;
b) optionally inducing cleavage of the molecular tags; and
c) detecting the presence or absence, or measuring the amount, of the molecular tags to detect and/or quantify the expression of the first cell surface moiety and of the second cell surface moiety in the sample.

In certain embodiments, the method according to the present disclosure can be used to measure co-expression of at least two different cell surface moieties in a single sample, preferably a patient sample. As such, it is particularly useful for predicting the response of a patient, preferably a cancer patient, to treatment with an agent or agents that bind the at least two different cell surface moieties. An example of such an agent is for instance a multispecific antibody.

In certain embodiments, the method used in the present disclosure includes a VeraTag® assay. The VeraTag® assay is well known in the art and is described in for instance WO 2017/161030 and references cited therein, which are incorporated herein in their entirety.

A VeraTag® assay can be performed in different formats. One format is a proximity assay using two primary binding molecules for each target moiety, wherein one of the primary binding molecules to each target moiety comprises a molecular tag and the other a cleavage inducible moiety.

In one embodiment, the present disclosure thus provides a method for detecting and/or quantifying expression in a sample of at least two cell surface moieties, comprising a first cell surface moiety and a second cell surface moiety, the method comprising:
a1) contacting a sample with a first and second binding molecule that specifically bind to a first moiety expressed on a cell surface and a third and fourth binding molecule that specifically bind to a second moiety expressed on a cell surface,
wherein the first binding molecule comprises a first molecular tag attached thereto via a cleavable linker and the second binding molecule comprises a cleavage inducing moiety, and wherein the third binding molecule comprises a second molecular tag attached thereto via a cleavable linker and the fourth binding molecule comprises a cleavage inducing moiety; or
a2) contacting a sample with a first and second binding molecule that specifically bind to a first moiety expressed on a cell surface and a third and fourth binding molecule that specifically bind to a second moiety expressed on a cell surface,
wherein the second binding molecule comprises a first molecular tag attached thereto via a cleavable linker and the first binding molecule comprises a cleavage inducing moiety, and wherein the fourth binding molecule comprises a second molecular tag attached thereto via a cleavable linker and the third binding molecule comprises a cleavage inducing moiety; or
a3) contacting a sample with a first and second binding molecule that specifically bind to a first moiety expressed on a cell surface and a third and fourth binding molecule that specifically bind to a second moiety expressed on a cell surface,
wherein the first binding molecule comprises a first molecular tag attached thereto via a cleavable linker and the second binding molecule comprises a cleavage inducing moiety, and wherein the fourth binding molecule comprises a second molecular tag attached thereto via a cleavable linker and the third binding molecule comprises a cleavage inducing moiety; or
a4) contacting a sample with a first and second binding molecule that specifically bind to a first moiety expressed on a cell surface and a third and fourth binding molecule that specifically bind to a second moiety expressed on a cell surface,
wherein the second binding molecule comprises a first molecular tag attached thereto via a cleavable linker and the first binding molecule comprises a cleavage inducing moiety, and wherein the third binding molecule comprises a second molecular tag attached thereto via a cleavable linker and the fourth binding molecule comprises a cleavage inducing moiety;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released first and second molecular tags to detect and/or quantify the expression of the first cell surface moiety and of the second cell surface moiety in the sample.

Another format is a proximity assay using two primary binding molecules for each target moiety and a secondary binding molecule against one of the primary binding molecules.

In one embodiment, the present disclosure thus provides a method for detecting and/or quantifying expression in a sample of at least two cell surface moieties, comprising a first cell surface moiety and a second cell surface moiety, the method comprising:
a1) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first and third binding molecules comprise a first and second molecular tag respectively, attached thereto via a cleavable linker, the fifth binding molecule binds to the second binding molecule and comprises a cleavage inducing moiety, and the sixth binding molecule binds to the fourth binding molecule and comprises a cleavage inducing moiety; or
a2) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second and fourth binding molecules comprise a first and second molecular tag respectively, attached thereto via a cleavable linker, the fifth binding molecule binds to the first binding molecule and comprises a cleavage inducing moiety, and the sixth binding molecule binds to the third binding molecule and comprises a cleavage inducing moiety; or
a3) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first and third binding molecules comprise a cleavage inducing moiety, the fifth binding molecule binds to the second binding molecule and comprises a first molecular tag attached thereto via a cleavable linker, and the sixth binding molecule binds to the fourth binding molecule and comprises a second molecular tag attached thereto via a cleavable linker; or
a4) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second and fourth binding molecules comprise a cleavage inducing moiety, the fifth binding molecule binds to the first binding molecule and a first molecular tag attached thereto via a cleavable linker, and the sixth binding molecule binds to the third binding molecule and comprises a second molecular tag attached thereto via a cleavable linker; or
a5) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding molecule comprises a first molecular tag attached thereto via a cleavable linker, the fourth binding molecule comprises a second molecular tag attached thereto via a cleavable linker, the fifth binding molecule binds to the second binding molecule and comprises a cleavage inducing moiety, and the sixth binding molecule binds to the third binding molecule and comprises a cleavage inducing moiety; or
a6) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding molecule comprises a first molecular tag attached thereto via a cleavable linker, the third binding molecule comprises a cleavage inducing moiety, the fifth binding molecule binds to the second binding molecule and comprises a cleavage inducing moiety, and the sixth binding molecule binds to the fourth binding molecule and comprises a second molecular tag attached thereto via a cleavable linker; or
a7) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding molecule comprises a first molecular tag attached thereto via a cleavable linker, the fourth binding molecule comprises a cleavage inducing moiety, the fifth binding molecule binds to the second binding molecule and comprises a cleavage inducing moiety, and the sixth binding molecule binds to the third binding molecule and comprises a second molecular tag attached thereto via a cleavable linker; or
a8) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second binding molecule comprises a first molecular tag attached thereto via a cleavable linker, the third binding molecule comprises a second molecular tag attached thereto via a cleavable linker, the fifth binding molecule binds to the first binding molecule and comprises a cleavage inducing moiety, and the sixth binding molecule binds to the fourth binding molecule and comprises a cleavage inducing moiety; or
a9) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second binding molecule comprises a first molecular tag attached thereto via a cleavable linker, the third binding molecule comprises a cleavage inducing moiety, the fifth binding molecule binds to the first binding molecule and comprises a cleavage inducing moiety, and the sixth binding molecule binds to the fourth binding molecule and comprises second molecular tag attached thereto via a cleavable linker; or
a10) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second binding molecule comprises a first molecular tag attached thereto via a cleavable linker, the fourth binding molecule comprises a cleavage inducing moiety, the fifth binding molecule binds to the first binding molecule and comprises a cleavage inducing moiety, and the sixth binding molecule binds to the third binding molecule and comprises a second molecular tag attached thereto via a cleavable linker; or
a11) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding molecule comprises a cleavage inducing moiety, the third binding molecule comprises a second molecular tag attached thereto via a cleavable linker, the fifth binding molecule binds to the second binding molecule and comprises a first molecular tag attached thereto via a cleavable linker, and the sixth binding molecule binds to the fourth binding molecule and comprises a cleavage inducing moiety; or
a12) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding molecule comprises a cleavage inducing moiety, the fourth binding molecule comprises a second molecular tag attached thereto via a cleavable linker, the fifth binding molecule binds to the second binding molecule and comprises a first molecular tag attached thereto via a cleavable linker, and the sixth binding molecule binds to the third binding molecule and comprises a cleavage inducing moiety; or
a13) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the first binding and fourth binding molecules comprise a cleavage inducing moiety, the fifth binding molecule binds to the second binding molecule and comprises a first molecular tag attached thereto via a cleavable linker, and the sixth binding molecule binds to the third binding molecule and comprises a second molecular tag attached thereto via a cleavable linker; or
a14) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second binding molecule comprise a cleavage inducing moiety, the third binding molecule comprises a second molecular tag attached thereto via a cleavable linker, the fifth binding molecule binds to the first binding molecule and comprises a first molecular tag attached thereto via a cleavable linker, and the sixth binding molecule binds to the fourth binding molecule and comprises a cleavage inducing moiety; or
a15) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second binding molecule comprise a cleavage inducing moiety, the fourth binding molecule comprises a second molecular tag attached thereto via a cleavable linker, the fifth binding molecule binds to the first binding molecule and comprises a first molecular tag attached thereto via a cleavable linker, and the sixth binding molecule binds to the third binding molecule and comprises a cleavage inducing moiety; or
a16) contacting a sample with a first and second binding molecule that specifically bind to a first moiety expressed on a cell surface, a third and fourth binding molecule that specifically bind to a second moiety expressed on a cell surface, a fifth binding molecule, and a sixth binding molecule,
wherein the second and third binding molecules comprise a cleavage inducing moiety, the fifth binding molecule binds to the first binding molecule and comprises a first molecular tag attached thereto via a cleavable linker, and the sixth binding molecule binds to the fourth binding molecule and comprises a second molecular tag attached thereto via a cleavable linker;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released first and second molecular tags to detect and/or quantify the expression of the first cell surface moiety and of the second cell surface moiety in the sample.

Another format is a proximity assay using two primary binding molecules for each target moiety and secondary binding molecules against each of the primary binding molecules, wherein one of the secondary binding molecules for each target moiety comprises a molecular tag and the other a cleavage inducible moiety.

In one embodiment, the present disclosure thus provides a method for detecting and/or quantifying expression in a sample of at least two cell surface moieties, comprising a first cell surface moiety and a second cell surface moiety, the method comprising:
a1) contacting a sample with a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,
wherein the fifth binding molecule binds to the first binding molecule and comprises a first molecular tag attached thereto via a cleavable linker, the seventh binding molecule binds to the second binding molecule and comprises a cleavage inducing moiety, the sixth binding molecule binds to the third binding molecule and comprises a second molecular tag attached thereto via a cleavable linker, and the eighth binding molecule binds to the fourth binding molecule and comprises a cleavage inducing moiety; or
a2) contacting a sample with a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,
wherein the fifth binding molecule binds to the first binding molecule and comprises a cleavage inducing moiety, the seventh binding molecule binds to the second binding molecule and comprises a first molecular tag attached thereto via a cleavable linker, the sixth binding molecule binds to the third binding molecule and comprises a cleavage inducing moiety, and the eighth binding molecule binds to the fourth binding molecule and comprises a second molecular tag attached thereto via a cleavable linker; or
a3) contacting a sample with a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,
wherein the fifth binding molecule binds to the first binding molecule and comprises a first molecular tag attached thereto via a cleavable linker, the seventh binding molecule binds to the second binding molecule and comprises a cleavage inducing moiety, the sixth binding molecule binds to the third binding molecule and comprises a cleavage inducing moiety, and the eighth binding molecule binds to the fourth binding molecule and comprises a second molecular tag attached thereto via a cleavable linker; or
a4) contacting a sample with a first and second binding molecule that specifically binds to a first moiety expressed on a cell surface, a third and fourth binding molecule that specifically binds to a second moiety expressed on a cell surface, a fifth binding molecule, a sixth binding molecule, a seventh binding molecule, and an eighth binding molecule,
wherein the fifth binding molecule binds to the first binding molecule and comprises a cleavage inducing moiety, the seventh binding molecule binds to the second binding molecule and comprises a first molecular tag attached thereto via a cleavable linker, the sixth binding molecule binds to the third binding molecule and comprises a second molecular tag attached thereto via a cleavable linker, and the eighth binding molecule binds to the fourth binding molecule and comprises a cleavage inducing moiety;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released first and second molecular tags to detect and/or quantify the expression of the first cell surface moiety and of the second cell surface moiety in the sample.

Another format is a DTT-mediated release, wherein the method for detecting and/or quantifying expression of at least a first cell surface moiety and of a second cell surface moiety in a sample comprises:
a) contacting a sample with at least one binding molecule that detects a first cell surface moiety and at least one binding molecule that detects a second cell surface moiety,
wherein at least one binding molecule that detects the first cell surface moiety and at least one binding molecule that detects the second cell surface moiety comprise a molecular tag attached thereto via a cleavable linker, and
wherein the molecular tag attached to the binding molecule that detects the first cell surface moiety is different from the molecular tag attached to the binding molecule that detects the second cell surface moiety;
b) inducing cleavage of the molecular tags; and
c) detecting the presence or absence of released molecular tags to detect and/or quantify the expression of the first cell surface moiety and of the second cell surface moiety in the sample.

In one format, the DTT-mediated release assay uses a primary binding molecule for each target moiety that comprises a molecular tag.

In one embodiment, the present disclosure thus provides a method for detecting and/or quantifying expression in a sample of at least two cell surface moieties, comprising a first cell surface moiety and a second cell surface moiety, the method comprising:
a) contacting a sample with a first binding molecule that specifically binds to a first moiety expressed on a cell surface and a second binding molecule that specifically binds to a second moiety expressed on a cell surface,
wherein the first binding molecule comprises a first molecular tag attached thereto via a cleavable linker; and
wherein the second binding molecule comprises a second molecular tag attached thereto via a cleavable linker;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released molecular tags to detect and/or quantify the expression of the first cell surface moiety and of the second cell surface moiety in the sample.

Another format is a DTT-mediated release assay using a primary binding molecule and a secondary binding molecule for each target moiety, wherein the secondary binding molecules comprise a molecular tag.

In one embodiment, the present disclosure thus provides a method for detecting and/or quantifying expression in a sample of at least two cell surface moieties, comprising a first cell surface moiety and a second cell surface moiety, the method comprising:
a) contacting a sample with a first binding molecule that specifically binds to a first moiety expressed on a cell surface, a second binding molecule that specifically binds to a second moiety expressed on a cell surface, a third binding molecule that specifically binds to the first binding molecule, and a fourth binding molecule that specifically binds to the second binding molecule,
wherein the third binding molecule comprises a first molecular tag attached thereto via a cleavable linker; and
wherein the fourth binding molecule comprises a second molecular tag attached thereto via a cleavable linker;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released molecular tags to detect and/or quantify the expression of the first cell surface moiety and of the second cell surface moiety in the sample.

Another format is a DTT-mediated release assay using a primary binding molecule for each target moiety, and a secondary binding molecule that binds to one of the primary binding molecules and which comprises a molecular tag.

In one embodiment, the present disclosure thus provides a method for detecting and/or quantifying expression in a sample of at least two cell surface moieties, comprising a first cell surface moiety and a second cell surface moiety, the method comprising:
a1) contacting a sample with a first binding molecule that specifically binds to a first moiety expressed on a cell surface, a second binding molecule that specifically binds to a second moiety expressed on a cell surface, and a third binding molecule that specifically binds to the first binding molecule,
wherein the second binding molecule comprises a first molecular tag attached thereto via a cleavable linker; and
wherein the third binding molecule comprises a second molecular tag attached thereto via a cleavable linker; or
a2) contacting a sample with a first binding molecule that specifically binds to a first moiety expressed on a cell surface, a second binding molecule that specifically binds to a second moiety expressed on a cell surface, and a third binding molecule that specifically binds to the second binding molecule,
wherein the first binding molecule comprises a first molecular tag attached thereto via a cleavable linker; and
wherein the third binding molecule comprises a second molecular tag attached thereto via a cleavable linker;
wherein the molecular tag attached to the binding molecule that detects the first cell surface moiety is different from the molecular tag attached to the binding molecule that detects the second cell surface moiety;
b) inducing cleavage of the first and second molecular tags; and
c) detecting the presence or absence of released molecular tags to detect and/or quantify the expression of the first cell surface moiety and of the second cell surface moiety in the sample.

In each of the exemplified formats above where a primary binding molecule and a secondary binding molecule is used, one or more binding molecules may be present between the primary and secondary binding molecules.

The method according to the present disclosure may comprise a combination of a proximity assay and a DTT-mediated release assay, wherein a proximity assay is used to detect a first cell surface moiety and a DTT-mediated release assay is used to detect a second cell surface moiety.

The method according to the present disclosure can be used to measure co-expression of at least two different cell surface moieties in a single sample. The knowledge gained therefrom can be used to predict the responsiveness of a patient, in particular a cancer patient, to an agent or agents binding the two different cell surface moieties.

The present disclosure thus provides a method for predicting the responsiveness of a patient, in particular a cancer patient, to an agent or agents binding at least a first cell surface moiety and a second cell surface moiety, in particular a moiety expressed on an immune effector cell and a moiety expressed on a tumor cell, the method comprising:
a) detecting the expression levels of a first cell surface moiety and a second cell surface member in a biological sample from a subject, in particular from a subject's tumor, using the method according to the present disclosure;
b) determining whether the expression levels of the first cell surface moiety and the second cell surface moiety in the subject's sample is above or below a threshold level; and
c) predicting that the subject is likely to respond to an agent or agents binding the first cell surface moiety and the second cell surface moiety if the expression levels of the first cell surface moiety and the second cell surface moiety in the subject's sample is equal to or above the threshold level. The agent or agents comprise an agent as defined further herein. This method is also referred to herein as “prediction method”.

The present disclosure also provides a method for treating a subject in need thereof, in particular a subject having cancer, the method comprising:
a) predicting responsiveness of a subject to an agent or agents binding a first cell surface moiety and a second cell surface moiety using the prediction method as described above; and
b) administering an agent or agents binding the first cell surface moiety and the second cell surface to a subject that is likely to respond according to the prediction. The agent or agents comprise an agent as defined further herein.

The present disclosure further provides an agent or agents binding a first cell surface moiety and a second cell surface moiety for the treatment of a subject, in particular a subject having cancer, wherein the treatment comprises:
a) predicting responsiveness of a subject to an agent or agents binding a first cell surface moiety and a second cell surface moiety using the prediction method as described above; and
b) administering an agent or agents binding the first cell surface moiety and the second cell surface to a subject that is likely to respond according to the prediction. The agent or agents comprise an agent as defined further herein.

The present disclosure further provides a method for detecting and/or quantifying the presence in a sample of clustering of at least two cell surface moieties, comprising a first cell surface moiety and a second cell surface moiety, the method comprising detecting the presence or absence of a signal, which signal is not detected unless the first and second cell surface moieties are in proximity of each other, in a sample in which the first and second cell surface moieties have been exposed to an agent having binding specificity for at least the first and second cell surface moieties. This method can be used to show if two or more cell surface moieties are in proximity of each other. When two or more cell surface moieties are in proximity of each other they are consider to cluster if it generates a signal as described herein. The proximity of the at least two cell surface moieties is caused or induced by an agent having binding specificity for the at least two cell surface moieties, such as for instance a multispecific antibody. In certain embodiments, the method can thus also be defined as comprising detecting a signal produced when the first and second cell surface moieties are simultaneously bound by an agent having binding specificity for the at least two cell surface moieties.

The method according to the present disclosure can be performed in different ways, including different formats of a proximity assay. One way of performing the method involves quenching of a signal from a fluorophore attached to a binding molecule that detects one of the cell surface moieties, wherein the quenching is caused by a quencher attached to a binding molecule that detects another of the cell surface moieties. Another way of performing the method involves a signal produced by the interference of a fluorophore attached to a binding molecule that detects one of the cell surface moieties with another, different, fluorophore attached to a binding molecule that detects another of the cell surface moieties.

In one format of a proximity assay, two primary binding molecules are used, one for each target moiety, wherein one of the primary binding molecules comprises a molecular tag and the other primary binding molecule comprises a cleavage inducible moiety.

In one embodiment, the present disclosure thus provides a method for detecting and/or quantifying the presence in a sample of clustering of at least two cell surface moieties, comprising a first cell surface moiety and a second cell surface moiety, wherein the method comprises:
a1) contacting a sample in which the first and second cell surface moieties have been exposed to an agent having binding specificity for at least the first and second cell surface moieties with a first binding molecule that specifically binds to the first cell surface moiety and a second binding molecule that specifically binds to the second cell surface moiety,
wherein the first binding molecule comprises a molecular tag attached thereto via a cleavable linker and the second binding molecule comprises a cleavage inducing moiety; or
a2) contacting a sample in which the first and second cell surface moieties have been exposed to an agent having binding specificity for at least the first and second cell surface moieties with a first binding molecule that specifically binds to the first cell surface moiety and a second binding molecule that specifically binds to the second cell surface moiety,
wherein the second binding molecule comprises a molecular tag attached thereto via a cleavable linker and the first binding molecule comprises a cleavage inducing moiety; and
b) inducing cleavage of the molecular tag; and
c) detecting the presence or absence of released molecular tag to detect and/or quantify the clustering of the first cell surface moiety with the second cell surface moiety in the sample.

In another format, a primary binding molecule is used for each target moiety and a secondary binding molecule against one of the primary binding molecules, wherein the primary binding molecule not bound by the secondary binding molecule comprises a molecular tag and the secondary binding molecule comprises a cleavage inducible moiety, or the primary binding molecule not bound by the secondary binding molecule comprises a cleavage inducing moiety and the secondary binding molecule comprises a molecular tag.

In one embodiment, the present disclosure thus provides a method for detecting and/or quantifying the presence in a sample of clustering of at least two cell surface moieties, comprising a first cell surface moiety with a second cell surface moiety, wherein the method comprises:
a1) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, and a third binding molecule,
wherein the first binding molecule comprises a molecular tag attached thereto via a cleavable linker and the third binding molecule binds to the second binding molecule and comprises a cleavage inducing moiety; or
a2) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, and a third binding molecule,
wherein the second binding molecule comprises a molecular tag attached thereto via a cleavable linker and the third binding molecule binds to the first binding molecule and comprises a cleavage inducing moiety; or
a3) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, and a third binding molecule,
wherein the first binding molecule comprises a cleavage inducing moiety and the third binding molecule binds to the second binding molecule and comprises a molecular tag attached thereto via a cleavable linker; or
a4) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, and a third binding molecule,
wherein the second binding molecule comprises a cleavage inducing moiety and the third binding molecule binds to the first binding molecule and comprises a molecular tag attached thereto via a cleavable linker;
b) inducing cleavage of the molecular tag; and
c) detecting the presence or absence of released molecular tag to detect and/or quantify the clustering of the first cell surface moiety with the second cell surface moiety in the sample.

In another format, a primary binding molecule is used for each target moiety and secondary binding molecules against both of the primary binding molecules, wherein one of the secondary binding molecules comprises a molecular tag and the other a cleavage inducible moiety.

In one embodiment, the present disclosure thus provides a method for detecting and/or quantifying the presence in a sample of clustering of at least two cell surface moieties, comprising a first cell surface moiety and a second cell surface moiety, wherein the method comprises:
a1) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, a third binding molecule, and a fourth binding molecule,
wherein the third binding molecule binds to the first binding molecule and comprises a molecular tag attached thereto via a cleavable linker, and the fourth binding molecule binds to the second binding molecule and comprises a cleavage inducing moiety; or
a2) contacting a sample in which the first and second cell surface moieties have been exposed to an agent having binding specificity for at least the first and second cell surface moieties with a first binding molecule that specifically binds to the first cell surface moiety, a second binding molecule that specifically binds to the second cell surface moiety, a third binding molecule, and a fourth binding molecule,
wherein the third binding molecule binds to the first binding molecule and comprises a cleavage inducing moiety; and the fourth binding molecule binds to the second binding molecule and comprises a molecular tag attached thereto via a cleavable linker,
b) inducing cleavage of the molecular tag; and
c) detecting released molecular tag to detect and/or quantify the clustering of the first cell surface moiety with the second cell surface moiety in the sample.

In each of the exemplified formats above where a primary binding molecule and a secondary binding molecule is used, one or more binding molecules may be present between the primary and secondary binding molecules.

The present disclosure further provides a method for detecting and/or quantifying the presence in a sample of clustering of at least two cell surface moieties, comprising a first cell surface moiety and a second cell surface moiety, the method comprising:
contacting a sample in which the first and a third cell surface moiety have been exposed to an agent having binding specificity for at least the first and third cell surface moieties with a first binding molecule that specifically binds to the first cell surface moiety, a second binding molecule that specifically binds to the second cell surface moiety, wherein at least one of the first binding molecule and the second binding molecule comprises a molecular tag which is not detected unless the first and second cell surface moieties are in proximity of each other; and
detecting the presence or absence of the molecular tag to detect the presence in the sample of clustering of the first cell surface moiety and the second cell surface moiety.

In this context, the at least first and third cell surface moieties are preferably different cell surface moieties. In certain embodiments, the first cell surface moiety is CD137 or another co-stimulatory molecule and the third cell surface moiety is a moiety on another cell, such as a tumor-associated moiety or an immune checkpoint moiety, preferably PD-L1. The first and second cell surface moieties are preferably the same cell surface moieties. In certain embodiments, the first and second cell surface moieties are CD137.

The method according to the present disclosure can be used to measure clustering, of two or more different cell surface moieties in a single sample, and in accordance with the present disclosure, in particular when the clustering is induced by an agent having binding specificity for the two or more different cell surface moieties. In certain embodiments, the knowledge gained therefrom can be used to determine if treatment with an agent having binding specificity for the two or more different cell surface moieties is effective or not, which determination is based on confirmation of the simultaneous binding of the agent having binding specificity for the two different cell surface moieties to those moieties.

The agent can be any single moiety that is capable of simultaneously binding to at least two cell surface moieties. In certain embodiments, the agent comprises at least a binding domain that specifically binds to a first cell surface moiety and a binding domain that specifically binds to a second cell surface moiety. Suitable agents for instance include binding molecules such as antibodies, including multispecific antibodies such as for instance bispecific and trispecific antibodies, antibody fragments, molecules comprising antibody-derived domains, and fusion proteins. The agent can also be referred to as a drug.

The present disclosure thus provides a method for determining the effectiveness of an agent, the agent comprising at least a binding domain that specifically binds to a first cell surface moiety and a binding domain that specifically binds to a second cell surface moiety, the method comprising detecting and/or quantifying clustering of a first cell surface moiety with a second cell surface moiety in a biological sample of a subject under treatment with the agent by using the method according to the present disclosure. The agent is an agent as defined further herein. This method is also referred to herein as “monitoring method”.

The present disclosure also provides a method for confirming a mode of action of an agent, the agent comprising at least a binding domain that specifically binds to at least a first cell surface moiety and a binding domain that specifically binds to a second cell surface moiety, the method comprising detecting and/or quantifying clustering of a first cell surface moiety with a second cell surface moiety in a biological sample of a subject under treatment with the agent by using the method according to the present disclosure. The agent is an agent as defined further herein. The mode of action is for instance simultaneous binding of an agent to the first and second cell surface moieties. In this context, the first and second cell surface moieties are preferably different cell surface moieties, and the agent is a multispecific antibody. Another mode of action is for instance the clustering of two or more cell surface moieties. In this context, in one embodiment, the at least first and second cell surface moieties are the same, and the agent is a monospecific antibody; and in another embodiment, the at least first and second cell surface moieties are different cell surface moieties, and the agent is a multispecific antibody. This method is also referred to herein as “confirmation method”. For instance, it can be determined if an agent, preferably a multispecific antibody having specificity for a cell surface moiety expressed on an immune effector cell, preferably CD137 or any other immune effector cell co-stimulatory moiety, and a cell surface moiety expressed on a tumor cell, preferably PD-L1 or any other tumor-associated moiety or immune checkpoint, induces clustering of two or more of the cell surface moieties expressed on the immune effector cell, preferably one or more CD137, or any other immune effector cell co-stimulatory, proteins.

The present disclosure further provides a method for treating a subject in need thereof, in particular a subject having cancer, the method comprising:
a) treating a subject with an agent binding a first cell surface moiety and a second cell surface moiety;
b) analyzing the effectiveness of the agent using the monitoring method as described herein, or confirming the mode of action of the agent using the confirmation method as described herein. The agent is an agent as defined further herein. In certain embodiments, this method may further comprise continuing or adapting the treatment based on the outcome of the analysis or confirmation, wherein adapting includes, but is not limited to, increasing or lowering the dose and/or increasing or lowering the frequency of administration of the agent, or ending treatment.

The present disclosure further provides an agent binding a first cell surface moiety and a second cell surface moiety for use in the treatment of a subject, in particular a subject having cancer, wherein the treatment comprises:
a) treating a subject with an agent binding a first cell surface moiety and a second cell surface moiety;
b) analyzing the effectiveness of the agent using the monitoring method as described herein, or confirming the mode of action of the agent using the confirmation method as described herein. The agent is an agent as defined further herein. In certain embodiments, the treatment may further comprise continuing or adapting the treatment based on the outcome of the analysis or confirmation, wherein adapting includes, but is not limited to, increasing or lowering the dose and/or increasing or lowering the frequency of administration of the agent, or ending treatment.

The method according to the present disclosure can further be used to screen one or more test agents for the ability to induce clustering of at least a first cell surface moiety with a second cell surface moiety.

The present disclosure thus further provides a method for screening one or more test agents for the ability to induce clustering of at least a first cell surface moiety with a second cell surface moiety, the method comprising:
a) contacting one or more test cell cultures with a test agent,
wherein the test cell culture comprises a cell expressing at least a first cell surface moiety and a second cell expressing a second cell surface moiety;
b) detecting the level of clustering of the first and second cell surface moieties using a method according to the present disclosure; and
c) comparing the level of clustering detected in step b) with the level of clustering detected for the clustering in a control cell culture not contacted with the test agent or contacted with a reference agent,
wherein the control cell culture comprises the first cell surface moiety and the second cell surface moiety. In certain embodiments, this method may further comprise selecting a test agent that induces an equal or higher level of clustering than the level of clustering in the control cell culture.

This method can be used to identify new, further or alternative binding molecules with binding specificity to two or more different cell surface moieties in addition to those already known. For instance, this method can be used to identify further or alternative binding molecules with binding specificity to CD137, or any other immune effector cell co-stimulatory moiety, and PD-L1, or any other tumor-associated moiety or immune checkpoint moiety.

The present disclosure further provides a kit of parts. In certain embodiments, the kit comprises the binding molecules that specifically bind to the at least first and second cell surface moieties in accordance with the methods as described herein. In one embodiment, the kit of parts comprises at least two binding molecules that specifically bind to a first and second cell surface moiety, optionally wherein one of the binding molecules comprises a molecular tag attached thereto via a cleavable linker and the other binding molecule comprises a cleavage inducing moiety, and instructions to contact a patient sample with the at least two binding molecules, optionally to induce cleavage of the molecular tag; and to measure the signal induced by contacting the patient sample with the at least two binding molecules.

The following is a further description of the features of the methods as described herein. For the purpose of clarity and a concise description, features are described herein as part of the same or separate embodiments, however, it will be appreciated that the scope of the present disclosure may include embodiments having combinations of all or some of the features described.

The method according to the present disclosure can be used to detect the expression of a first cell surface moiety and a second cell surface moiety. The present disclosure further provides a method that can be used to show if two cell surface moieties are in proximity of each other. The detection methods used, the release of a molecular tag from a binding molecule bound to (one of) the cell surface moieties, also allows for the quantification of the expression of the (complex of the) first and second cell surface moieties.

In certain embodiments, the method according to the present disclosure allows for the detection and/or quantification of a first and second cell surface moiety in a single sample. For this, the molecular tag attached to the antibody binding the first cell surface moiety is different from the molecular tag attached to the antibody binding the second cell surface moiety. In certain embodiments, the method can be used to determine if the first and second cell moieties are co-expressed in a certain sample.

The sample in the methods according to certain embodiments of the present disclosure can be, but is not limited to, a tissue sample, a blood sample, or cultured cells. Preferably, the sample is a tissue sample, blood sample or cultured cells from a subject or patient. A tissue sample from a subject or patient can be a fresh sample or a formalin-fixed paraffin-embedded (FFPE), or otherwise fixed, sample. The methods are particularly useful for the detection and/or quantification of a (cluster of a) first and second cell surface moiety in a tumor biopsy sample from a subject having cancer.

In certain embodiments, the first cell surface moiety and the second cell surface moiety are preferably different moieties and can be expressed on the same cell type, such as for instance a tumor cell or an immune cell, on the same cell, or on different cells. In certain embodiments, the first cell surface moiety and the second cell surface moiety are preferably expressed on different cell types.

In certain embodiments, the methods of the present disclosure are useful in any application where it is of interest to measure the co-expression of two or more cell surface moieties, and/or where the determination of clustering of two or more cell surface moieties on the same or separate cells is of interest.

In certain embodiments, one of the at least two cell surface moieties is preferably expressed on an immune effector cell, in particular a NK cell, a T cell, a B cell, a monocyte, a macrophage, a dendritic cell or a neutrophilic granulocyte, preferably a T cell.

In certain embodiments, one of the at least two cell surface moieties is expressed on a cell which can be from a tumor or from an immune cell origin, such as for instance but not limited to a tumor cell, B cell, myeloid cell, dendritic cell, or neutrophil.

In certain embodiments, one of the at least two cell surface moieties is expressed on an immune effector cell, in particular a NK cell, a T cell, a B cell, a monocyte, a macrophage, a dendritic cell or a neutrophilic granulocyte, preferably a T cell, and the other of the at least two cell surface moieties is expressed on a cell which can be from a tumor or from an immune cell origin, such as for instance but not limited to a tumor cell, B cell, myeloid cell, dendritic cell, or neutrophil.

In certain embodiments an immune effector cell can be an NK cell, a T cell, a B cell, a monocyte, a macrophage, a dendritic cell or a neutrophilic granulocyte, preferably a T cell.

In certain embodiments, an immune effector cell co-stimulatory moiety can be CD137, OX40, GITR, CD27, CD28, ICOS, CD40L or LIGHT, preferably CD137.

In certain embodiments, immune checkpoint moieties or tumor associated moieties can be selected from, but are not limited to PD-L1, PD-L2, B7-H3, B7-H4, TIM3, CD47 or CD70, preferably PD-L1.

The first and second cell surface moieties can be any cell surface moieties that cluster in response to an agent that brings both cell surface moieties in proximity of each other. In certain embodiments, the first and second cell surface moieties can be the same and the method is used to detect and/or quantify the clustering, for instance dimerization or trimerization, of these cell surface moieties in response to an agent. This is for instance exemplified herein for the clustering of at least two CD137 molecules.

In certain embodiments, the first cell surface moiety is CD137 and the second cell surface moiety is PD-L1.

Methods for measuring HER2 homodimers, as well as HER1/HER2 heterodimers, HER2/HER3 heterodimers, HGF-c-Met complex, HER3-PI3K complex, PD-1-PD-L1 complex, are not part of the present disclosure.

The present disclosure thus provides a method for detecting and/or quantifying expression in a sample of at least two cell surface moieties, comprising a first cell surface moiety and a second cell surface moiety, as described herein, such that the first and second cell surface moieties are not HER2; HER1 and HER2; HER2 and HER3; HGF and c-Met; HER3 and PI3K; or PD-1 and PD-L1.

The present disclosure also provides a method for detecting and/or quantifying detecting the presence in a sample of clustering of at least two cell surface moieties, comprising a first cell surface moiety and a second cell surface moiety, as described herein, such that the first and second cell surface moieties are not HER2; HER1 and HER2; HER2 and HER3; HGF and c-Met; HER3 and PI3K; or PD-1 and PD-L1.

In certain embodiments of the methods of the present disclosure, the sample is contacted with binding molecules that bind to the first and second cell surface moieties. These are also referred to as assay binding molecules.

In certain formats of the method according to the present disclosure, two binding molecules are used for each moiety. The present disclosure refers to the two binding molecules that bind to the first cell surface moiety as a first binding molecule and a second binding molecule. The two binding molecules that bind to the second cell surface moiety are referred to herein as a third binding molecule and a fourth binding molecule. The numbers used in referring to these binding molecules indicate that the binding molecules are different from one another with respect to binding specificity and/or for easier visualization of the examples of useful assay formats. The numbers do not refer to any particular order or required presence of one or more of the binding molecules. Also, in the formats using primary binding molecules and one or more secondary binding molecules, other binding molecules may be present between the primary binding molecules, i.e. the binding molecules that bind directly to the cell surface moieties, and the secondary binding molecules, i.e. the binding molecules comprising a molecular tag or cleavage inducing moiety.

In certain formats of the method according to the present disclosure, the two binding molecules that bind a first cell surface moiety are binding molecules that bind different epitopes, and are selected such that they do not interfere with each other's binding to the first cell surface moiety. Similarly, the two binding molecules that bind a second cell surface moiety are binding molecules that bind different epitopes, and are selected such that they do not interfere with each other's binding to the second cell surface moiety. The first, second, third, and/or fourth binding molecule can bind an extracellular domain of the cell surface moiety; but it is also possible that they bind an intracellular domain of the cell surface moiety. A combination thereof is also possible. For instance, the two binding molecules that bind to the first cell surface moiety may be directed to an extracellular domain thereof, whereas the two binding molecules that bind to the second cell surface moiety may bind to an intracellular domain thereof; or one of the binding molecules that binds to the first cell surface moiety may bind to an extracellular domain and the other may bind an intracellular domain thereof, and the same for the second cell surface moiety; or one of the binding molecules that binds to the first cell surface moiety may bind to an extracellular domain and the other may bind an intracellular domain thereof, while both binding molecules that bind to the second cell surface moiety may bind to the extracellular or intracellular domain thereof, or vice versa.

In certain embodiments, one primary binding molecule is used for each moiety. The present disclosure refers to the two primary binding molecules that bind to the first cell surface moiety and second cell surface moiety as a first binding molecule and a second binding molecule. The numbers used in referring to these binding molecules indicate that the binding molecules are different from one another with respect to binding specificity and/or for easier visualization of the examples of useful assay formats. The numbers do not refer to any particular order or required presence of one or more of the binding molecules. They also do not indicate that they would be the same as the first and second binding molecules referred to in relation to other embodiments of the present disclosure. Also, in the formats using primary binding molecules and one or more secondary binding molecules, other binding molecules may be present between the primary binding molecules, i.e. the binding molecules that bind directly to the cell surface moieties, and the secondary binding molecules, i.e. the binding molecules comprising a molecular tag or cleavage inducing moiety.

In certain embodiments, the two binding molecules that bind a first and second cell surface moiety are binding molecules that bind different cell surface moieties. The first and second binding molecules can bind an extracellular domain of the cell surface moiety; but it is also possible that they bind an intracellular domain of the cell surface moiety. A combination thereof is also possible. For instance, the binding molecule that bind to the first cell surface moiety may bind to an extracellular domain thereof, whereas the binding molecule that binds to the second cell surface moiety may bind to an intracellular domain thereof, or vice versa.

The binding molecules used in certain embodiments of the methods according to the present disclosure are preferably antibodies, or antigen-binding fragments thereof. In this context, the first and second cell surface moieties can be considered antigens. Antibodies, or antigen-binding fragments thereof, that specifically bind to an antigen are known in the art and are available for a large number of different antigens. They are commercially available or can readily be produced. Such antibodies, or antigen-binding fragments thereof, usually bind to the antigen but do not otherwise exhibit a biological function, such as for instance blocking an interaction between the antigen and its ligand, or inducing cell killing activity.

In certain formats of the method according to the present disclosure, one of the primary binding molecules against each cell surface moiety comprises a molecular tag or a cleavage inducing moiety. In certain formats of the method of the present disclosure, one of the primary binding molecules comprises a molecular tag and the other a cleavage inducing moiety. Such a molecular tag or a cleavage inducing moiety can be attached to the primary binding molecule, in particular an antibody, using standard techniques in the art. In some instances it may be difficult to attach a molecular tag or cleavage inducing moiety to a certain binding molecule. In that case, one can use a secondary binding molecule, usually an antibody, to which the molecular tag or cleavage inducing moiety is attached. Such secondary binding molecules comprising a molecular tag or cleavage inducing moiety are typically directed to the Fc region of the primary binding molecule, and are generally commercially available.

The molecular tag can be any molecular moiety, such as a molecule, that can be detected. In certain instances upon release, the molecular moiety provides a measurable signal. A molecular tag may be chosen based on one or more of its properties that distinguishes it from other moieties, the properties including, but not limited to, electrophoretic mobility, molecular weight, shape, solubility, pKa, hydrophobicity, charge, charge/mass ratio, and polarity. A difference in at least one of these properties allows the separation of molecular tags in an assay wherein multiple cell surface moieties are measured in a single sample. In certain embodiments, the molecular tag comprises a detection moiety, such as for instance, but not limited to a fluorescent label, a chromogenic label, a radioactive label, or an electrochemical label. Exemplary fluorescent dyes include water-soluble rhodamine dyes, fluoresceins, 4,7-dichlorofluoresceins, benzoxanthene dyes and energy transfer dyes, as disclosed in the following references: Handbook of Molecular Probes and Research Reagents, 8^{th} ed. (2002), Molecular Probes, Eugene, Oreg.; WO 2001/32783; U.S. Pat. Publ. Nos. US 2002-0081616, US 2002-0086985; and Lee et al., 1997, Nucleic Acids Research 25:2816-2822. Examples of suitable molecular tags include, but are not limited to, a VeraTag® reporter molecule. VeraTag® reporter molecules are well known in the art. Preferred molecular tags are for instance VeraTag® reporter molecules Pro11 and Pro125. Pro11 is an example of a light-releasable tag, where Pro125 is an example of a DTT-releasable tag. Other VeraTag® molecules are for instance described in U.S. Pat. Publ. Nos. US 2004-0166529; US 2004-0126818; US 2003-0013126; US 2005-0079565; and US 2011-0180408, each of which and the references cited therein are incorporated in their entireties herein.

The cleavage-inducing moiety can be any moiety capable of directly or indirectly inducing cleavage of the molecular tag from the binding molecule to which the molecular tag is attached via a cleavable linker. In certain embodiments, the cleavage-inducing moiety is for instance a moiety that produces an active species capable of cleaving a cleavable linker. Illustrative active species include singlet oxygen, hydrogen peroxide, NADH, and hydroxyl radicals, phenoxy radical, superoxide and the like. Illustrative quenchers for active species that cause oxidation include polyenes, carotenoids, vitamin E, vitamin C, amino acidpyrrole N-conjugates of tyrosine, histidine and glutathione. See, e.g., Beutner et al., 2000, Meth. Enzymol. 319:226-241. One example is wherein biotin conjugated to a binding molecule is contacted with streptavidin-conjugated methylene blue and exposed to light, resulting in the release of a singlet oxygen capable of cleaving a cleavable linker.

In certain assay formats, the molecular tag is attached to a binding molecule via a cleavable linker. A cleavable linker can be any cleavable linker, including, but not limited to, a linker that may be cleaved by singlet oxygen or hydrogen peroxide, or by DTT. A linker that can be cleaved by DTT is a SS-linker. Cleavable linkages may also include linkages that are labile to agents that operate throughout a reaction mixture, such as base-labile linkages, photocleavable linkages, linkages cleavable by reduction, linkages cleaved by oxidation, acid-labile linkages and peptide linkages cleavable by specific proteases. References describing many such linkages include Greene and Wuts, 1991, Protective Groups in Organic Synthesis, Second Edition, John Wiley & Sons, New York; Hennanson, 1996, Bioconjugate Techniques, Academic Press, New York; and U.S. Pat. Publ. No. US 2003-0119059.

Cleavage of the molecular tag from a binding molecule can be induced by methods known in the art, including, but not limited to the use of photo-induction and DTT-mediated release. Photo-induction induces the activation of a light-absorbing molecule that when activated by light converts molecular oxygen into singlet oxygen. Inducing cleavage by using DTT involves DTT-induced cleavage of a disulfide linker that is cleavable by reduction and that links a molecular tag to a binding molecule.

In certain embodiments, the signal that is measured in the methods of the present disclosure is the amount of molecular tags released. In certain formats of the methods of the present disclosure, at least two different molecular tags are used. In certain embodiments these can be separated before detection by methods known in the art, including, but not limited to, electrophoresis or methods based on a difference in molecular weight, shape, solubility, pKa, hydrophobicity, charge, charge/mass ratio, and polarity. The detection method depends on the molecular tag.

In the context of the present disclosure “proximity” means that the binding molecule comprising a molecular tag attached thereto and the binding molecule comprising a cleavage-inducing moiety are within a distance that allows for cleavage of the molecular tag induced by the cleavage-inducing moiety. In a VeraTag® assay this is about 1000 nm, preferably within about 20-200 nm or 30-100 nm of each other. Other ranges for proximity apply depending on the nature of the molecular tag used, and can be readily determined by a person skilled in the art and is information provided by suppliers of commercially available tags, quenchers and reporter moieties. The information needed for a person skilled in the art to apply a particular proximity assay in the present disclosure is available in the art. For instance Nathan P. 2020, Assay Guidance Manual, Compound-Mediated Assay Interferences in Homogenous Proximity Assays (herein incorporated by reference in its entirety), provides information on donor and acceptor fluorophores that can be used in, amongst others, FRET assays, including a description of optimal distances between donor and acceptor fluorophores (see for instance Tables 2 and 3).

The method according to the present disclosure can be used to measure co-expression of two different cell surface moieties in a single sample. The knowledge gained therefrom can be used to determine a patient's treatment plan. For instance, if two cell surface moieties are co-expressed in a patient's tissue or blood sample, it can be determined to treat the patient with an agent or agents that target these two cell surface moieties. One example of such a situation is where a tumor biopsy sample of a cancer patient shows co-expression of two tumor-associated antigens on tumor cells. The patient may then successfully be treated with one or more agents that bind these tumor-associated antigens and which interfere with the signaling pathway of the tumor-associated antigens and/or induce T cell-mediated tumor cell killing. If the patient's tumor sample does not show co-expression of such tumor-associated antigens, it may be determined by a treating physician that a patient is unlikely to benefit from such treatment. Another situation is for instance wherein the tumor-biopsy shows co-expression of a tumor-associated antigen on a tumor cell and an antigen expressed on immune effector cells. This indicates that immune effector cells, such as for instance T cells and/or NK cells, are present in the tumor microenvironment. Such patient may benefit from treatment with an agent that brings the immune cells in close proximity of the tumor cells and/or activates the immune effector cells such that the tumor cells will selectively be killed. In certain embodiments, the method of the present disclosure can thus be used to predict the response of a patient, preferably a cancer patient, to treatment with an agent or agents that bind two different cell surface moieties.

An example of an agent that binds two different cell surface moieties is for instance a multispecific antibody. Such multispecific antibody can be a bispecific, or trispecific, antibody or antigen-binding fragment thereof that simultaneously binds to both cell surface moieties. Such multispecific antibody can exhibit monovalent binding for both cell surface moieties, such that the multispecific antibody comprises a single antigen-binding fragment for each cell surface moiety. The cell surface moieties can be any of the first and second cell surface moieties as disclosed herein.

A specific example of a multispecific antibody that binds two different cell surface moieties, and in relation to which the method of the present disclosure is useful, is a multispecific antibody that binds to PD-L1 on tumor cells and CD137 on T cells. Such multispecific antibody may comprise a CD137 binding domain comprising a heavy chain CDR3 (HCDR3) with an amino acid sequence as set forth in any one of SEQ ID NO: 4, 8, 12, 16, 19, 23, 27, 30, 34, 38, 42, 45, 48, or 52, allowing for one, two, or three amino acid substitutions therein. In certain embodiments, the CD137 binding domain comprises a heavy chain CDR3 (HCDR3) with an amino acid sequence as set forth in any one of SEQ ID NO: 4, 8, 12, 16, 19, 23, 27, 30, 34, 38, 42, 45, 48, or 52. The CD137 binding domain may further comprise a heavy chain CDR1 (HCDR1) with an amino acid sequence as set forth in any one of SEQ ID NO: 2, 6, 10, 14, 18, 21, 25, 32, 36, 40, 44, or 50, allowing for one, two, or three amino acid substitutions therein, and/or a heavy chain CDR2 (HCDR2) with an amino acid sequence as set forth in any one of SEQ ID NO: 3, 7, 11, 15, 22, 26, 29, 33, 37, 41, 47, or 51, allowing for one, two, or three amino acid substitutions therein. In certain embodiments, the CD137 binding domain may comprise a heavy chain CDR1 (HCDR1) with an amino acid sequence as set forth in any one of SEQ ID NO: 2, 6, 10, 14, 18, 21, 25, 32, 36, 40, 44, or 50; and/or a heavy chain CDR2 (HCDR2) with an amino acid sequence as set forth in any one of SEQ ID NO: 3, 7, 11, 15, 22, 26, 29, 33, 37, 41, 47, or 51. Any combination of HCDR1, HCDR2, and HCDR3 is possible. Preferred CD137 binding domains comprise a combination of HCDR1, HCDR2, and HCDR3 of SEQ ID NO:2, SEQ ID NO:3, and SEQ ID NO:4; SEQ ID NO:6, SEQ ID NO:7, and SEQ ID NO:8; SEQ ID NO:10, SEQ ID NO:11, and SEQ ID NO:12; SEQ ID NO:14, SEQ ID NO:15, and SEQ ID NO:16; SEQ ID NO:18, SEQ ID NO:3, and SEQ ID NO:19; SEQ ID NO:21, SEQ ID NO:22, and SEQ ID NO:23; SEQ ID NO:25, SEQ ID NO:26, and SEQ ID NO:27; SEQ ID NO:10, SEQ ID NO:29, and SEQ ID NO:30; SEQ ID NO:32, SEQ ID NO:33, and SEQ ID NO:34; SEQ ID NO:36, SEQ ID NO:37, and SEQ ID NO:38; SEQ ID NO:40, SEQ ID NO:41, and SEQ ID NO:42; SEQ ID NO:44, SEQ ID NO:41, and SEQ ID NO:45; SEQ ID NO:2, SEQ ID NO:47, and SEQ ID NO:48; or SEQ ID NO:50, SEQ ID NO:52, and SEQ ID NO:52. The CD137 binding domain of such multispecific antibody may comprise a heavy chain variable region having any one of SEQ ID NO:1, 5, 9, 13, 17, 20, 24, 28, 31, 35, 39, 43, 46, or 49, or having at least 80%, 85%, 90%, 95%, or 99%, preferably 95%, sequence identity to the framework regions thereof. In certain embodiments, the CD137 binding domain of such multispecific antibody also comprises a CH1 domain. Any CH1 domain may be used. An example of a suitable CH1 domain is provided by the amino acid sequence provided as SEQ ID NO: 116.

The multispecific antibody may further comprise a PD-L1 binding domain comprising a heavy chain CDR3 (HCDR3) with an amino acid sequence as set forth in any one of SEQ ID NO: 56, 58, 61, 72, 76, 80, 84, 88, 91, 95, 99, 102, or 106, allowing for one, two, or three amino acid substitutions therein. In certain embodiments, the PD-L1 binding domain comprises a heavy chain CDR3 (HCDR3) with an amino acid sequence as set forth in any one of SEQ ID NO: 56, 58, 61, 72, 76, 80, 84, 88, 91, 95, 99, 102, or 106. The PD-L1 binding domain may further comprise a heavy chain CDR1 (HCDR1) with an amino acid sequence as set forth in any one of SEQ ID NO: 54, 60, 65, 68, 70, 74, 78, 82, 86, 90, or 93, allowing for one, two, or three amino acid substitutions therein, and/or a heavy chain CDR2 (HCDR2) with an amino acid sequence as set forth in any one of SEQ ID NO: 55, 3, 63, 66, 71, 75, 79, 83, 87, 94, 98, 101, 105, or 108, allowing for one, two, or three amino acid substitutions therein. In certain embodiments, the PD-L1 binding domain comprises a heavy chain CDR1 (HCDR1) with an amino acid sequence as set forth in any one of SEQ ID NO: 54, 60, 65, 68, 70, 74, 78, 82, 86, 90, or 93; and/or a heavy chain CDR2 (HCDR2) with an amino acid sequence as set forth in any one of SEQ ID NO: 55, 3, 63, 66, 71, 75, 79, 83, 87, 94, 98, 101, 105, or 108. Any combination of HCDR1, HCDR2, and HCDR3 is possible. Preferred PD-L1 binding domains comprise a combination of HCDR1, HCDR2, and HCDR3 of SEQ ID NO: 54, SEQ ID NO: 55, and SEQ ID NO: 56; SEQ ID NO: 54, SEQ ID NO: 55, and SEQ ID NO: 58; SEQ ID NO: 60, SEQ ID NO: 3, and SEQ ID NO: 61; SEQ ID NO: 60, SEQ ID NO: 63, and SEQ ID NO: 56; SEQ ID NO: 65, SEQ ID NO: 66, and SEQ ID NO: 56; SEQ ID NO: 68, SEQ ID NO: 55, and SEQ ID NO: 56; SEQ ID NO: 70, SEQ ID NO: 71, and SEQ ID NO: 72; SEQ ID NO: 74, SEQ ID NO: 75, and SEQ ID NO: 76; SEQ ID NO: 78, SEQ ID NO: 79, and SEQ ID NO: 80; SEQ ID NO: 82, SEQ ID NO: 83, and SEQ ID NO: 84; SEQ ID NO: 86, SEQ ID NO: 87, and SEQ ID NO: 88; SEQ ID NO: 90, SEQ ID NO: 79, and SEQ ID NO: 91; SEQ ID NO: 93, SEQ ID NO: 94, and SEQ ID NO: 95; SEQ ID NO: 68, SEQ ID NO: 55, and SEQ ID NO: 56; SEQ ID NO: 70, SEQ ID NO: 98, and SEQ ID NO: 99; SEQ ID NO: 93, SEQ ID NO: 101, and SEQ ID NO: 102; SEQ ID NO: 74, SEQ ID NO: 105, and SEQ ID NO: 106; or SEQ ID NO: 86, SEQ ID NO: 108, and SEQ ID NO: 88. The PD-L1 binding domain of such multispecific antibody may comprise a heavy chain variable region having any one of SEQ ID NO: 53, 57, 59, 62, 64, 67, 69, 73, 77, 81, 85, 89, 92, 96, 97, 100, 103, 104, or 107, or having at least 80%, 85%, 90%, 95%, 99%, preferably 95%, sequence identity to the framework regions thereof. In certain embodiments, the PD-L1 binding domain of such multispecific antibody also comprises a CH1 domain. Any CH1 domain may be used. An example of a suitable CH1 domain is provided by the amino acid sequence provided as SEQ ID NO: 116.

In certain embodiments, the multispecific antibody may comprise any combination of the CD137 and PD-L1 binding domains as disclosed herein, see for instance Table 1. One such multispecific antibody is MCLA-145.

In certain embodiments, the multispecific antibody may further comprise any light chain. An example of a suitable light chain comprises a light chain variable region comprising a light chain CDR3 (LCDR3) with an amino acid sequence as set forth in SEQ ID NO: 113, allowing for one, two, or three amino acid substitutions therein. In certain embodiments, the light chain variable region comprises a light chain CDR3 (LCDR3) with an amino acid sequence as set forth in SEQ ID NO: 113. The light chain variable region may further comprise a light chain CDR1 (LCDR1) with an amino acid sequence as set forth in SEQ ID NO: 111, allowing for one, two, or three amino acid substitutions therein, and/or a light chain CDR2 (LCDR2) with an amino acid sequence as set forth in SEQ ID NO: 112, allowing for one, two, or three amino acid substitutions therein. In certain embodiments, the light chain variable region comprises a light chain CDR1 (LCDR1) with an amino acid sequence as set forth in SEQ ID NO: 111; and/or a light chain CDR2 (LCDR2) with an amino acid sequence as set forth in SEQ ID NO: 112. The light chain variable region of the multispecific antibody may comprise a light chain variable region having SEQ ID NO: 110, or having at least 80%, 85%, 90%, 95%, 99% sequence identity to the framework regions thereof. In certain embodiments, the light chain of such multispecific antibody also comprises a CL domain. Any CL domain may be used. An example of a suitable CL domain is provided by the amino acid sequence provided as SEQ ID NO: 115.

In certain embodiments, the multispecific antibody may further comprise an Fc region or part thereof. Such Fc region may comprise any modification known in the art, such as for instance, but not limited to, modifications to eliminate or reduce Fc effector function, and/or modifications that promote heterodimerization of the different CH3 domains. Any Fc region may be used. An example of a suitable Fc region is provided by the amino acid sequences provided as SEQ ID NO: 116-120.

The method according to the present disclosure can be used to detect clustering of at least two different cell surface moieties in a single sample, and in accordance with the present disclosure, in particular when the clustering is induced by an agent having binding specificity for the at least two different cell surface moieties. The knowledge gained therefrom can be used to determine if a patient is benefitting from treatment with an agent having binding specificity for the two different cell surface moieties, and thus if treatment is to be continued, adapted, or terminated. For instance, if the treatment agent is administered but no clustering is observed or is below a certain threshold level it can be determined to end treatment. Or if some clustering, is observed but not at the desired level, it can be determined to increase the treatment dose and/or treatment interval. In certain embodiments, the method according to the present disclosure can thus be considered as a method to monitor the patient's response to a particular treatment.

An example of an agent having binding specificity for the two different cell surface moieties is for instance a multispecific antibody. Such multispecific antibody can be a bispecific, or trispecific, antibody or antigen-binding fragment thereof that simultaneously binds to both cell surface moieties. Such multispecific antibody can exhibit monovalent binding for both cell surface moieties, such that the multispecific antibody comprises a single antigen-binding fragment for each cell surface moiety. The method of the present disclosure can be used in any situation wherein two or more cell surface moieties are clustered by an agent having binding specificity for those two or more cell surface moieties. The agent having binding specificity for two different cell surface moieties can thus bind any cell surface moieties, such as those disclosed herein, but is not limited thereto.

A specific example of a multispecific antibody that binds two different cell surface moieties, and in relation to which the method of the present disclosure is useful, is a multispecific antibody that binds to PD-L1 on tumor cells and CD137 on T cells. Such multispecific antibody may comprise a CD137 binding domain comprising a heavy chain CDR3 (HCDR3) with an amino acid sequence as set forth in any one of SEQ ID NO: 4, 8, 12, 16, 19, 23, 27, 30, 34, 38, 42, 45, 48, or 52, allowing for one, two, or three amino acid substitutions therein. In certain embodiments, the CD137 binding domain comprises a heavy chain CDR3 (HCDR3) with an amino acid sequence as set forth in any one of SEQ ID NO: 4, 8, 12, 16, 19, 23, 27, 30, 34, 38, 42, 45, 48, or 52. The CD137 binding domain may further comprise a heavy chain CDR1 (HCDR1) with an amino acid sequence as set forth in any one of SEQ ID NO: 2, 6, 10, 14, 18, 21, 25, 32, 36, 40, 44, or 50, allowing for one, two, or three amino acid substitutions therein, and/or a heavy chain CDR2 (HCDR2) with an amino acid sequence as set forth in any one of SEQ ID NO: 3, 7, 11, 15, 22, 26, 29, 33, 37, 41, 47, or 51, allowing for one, two, or three amino acid substitutions therein. In certain embodiments, the CD137 binding domain comprises a heavy chain CDR1 (HCDR1) with an amino acid sequence as set forth in any one of SEQ ID NO: 2, 6, 10, 14, 18, 21, 25, 32, 36, 40, 44, or 50; and/or a heavy chain CDR2 (HCDR2) with an amino acid sequence as set forth in any one of SEQ ID NO: 3, 7, 11, 15, 22, 26, 29, 33, 37, 41, 47, or 51. Any combination of HCDR1, HCDR2, and HCDR3 is possible. Preferred CD137 binding domains comprise a combination of HCDR1, HCDR2, and HCDR3 of SEQ ID NO:2, SEQ ID NO:3, and SEQ ID NO:4; SEQ ID NO:6, SEQ ID NO:7, and SEQ ID NO:8; SEQ ID NO:10, SEQ ID NO:11, and SEQ ID NO:12; SEQ ID NO:14, SEQ ID NO:15, and SEQ ID NO:16; SEQ ID NO:18, SEQ ID NO:3, and SEQ ID NO:19; SEQ ID NO:21, SEQ ID NO:22, and SEQ ID NO:23; SEQ ID NO:25, SEQ ID NO:26, and SEQ ID NO:27; SEQ ID NO:10, SEQ ID NO:29, and SEQ ID NO:30; SEQ ID NO:32, SEQ ID NO:33, and SEQ ID NO:34; SEQ ID NO:36, SEQ ID NO:37, and SEQ ID NO:38; SEQ ID NO:40, SEQ ID NO:41, and SEQ ID NO:42; SEQ ID NO:44, SEQ ID NO:41, and SEQ ID NO:45; SEQ ID NO:2, SEQ ID NO:47, and SEQ ID NO:48; or SEQ ID NO:50, SEQ ID NO:52, and SEQ ID NO:52. The CD137 binding domain of such multispecific antibody may comprise a heavy chain variable region having any one of SEQ ID NO: 1, 5, 9, 13, 17, 20, 24, 28, 31, 35, 39, 43, 46, or 49, or having at least 80%, 85%, 90%, 95%, 99%, preferably 95%, sequence identity to the framework regions thereof. In certain embodiments, the CD137 binding domain of such multispecific antibody also comprises a CH1 domain. Any CH1 domain may be used. An example of a suitable CH1 domain is provided by the amino acid sequence provided as SEQ ID NO: 116.

The multispecific antibody may further comprise a PD-L1 binding domain comprising a heavy chain CDR3 (HCDR3) with an amino acid sequence as set forth in any one of SEQ ID NO: 56, 58, 61, 72, 76, 80, 84, 88, 91, 95, 99, 102, or 106, allowing for one, two, or three amino acid substitutions therein. In certain embodiments, the PD-L1 binding domain comprises a heavy chain CDR3 (HCDR3) with an amino acid sequence as set forth in any one of SEQ ID NO: 56, 58, 61, 72, 76, 80, 84, 88, 91, 95, 99, 102, or 106. The PD-L1 binding domain may further comprise a heavy chain CDR1 (HCDR1) with an amino acid sequence as set forth in any one of SEQ ID NO: 54, 60, 65, 68, 70, 74, 78, 82, 86, 90, or 93, allowing for one, two, or three amino acid substitutions therein, and/or a heavy chain CDR2 (HCDR2) with an amino acid sequence as set forth in any one of SEQ ID NO: 55, 3, 63, 66, 71, 75, 79, 83, 87, 94, 98, 101, 105, or 108, allowing for one, two, or three amino acid substitutions therein. In certain embodiments, the PD-L1 binding domain comprises a heavy chain CDR1 (HCDR1) with an amino acid sequence as set forth in any one of SEQ ID NO: 54, 60, 65, 68, 70, 74, 78, 82, 86, 90, or 93; and/or a heavy chain CDR2 (HCDR2) with an amino acid sequence as set forth in any one of SEQ ID NO: 55, 3, 63, 66, 71, 75, 79, 83, 87, 94, 98, 101, 105, or 108. Any combination of HCDR1, HCDR2, and HCDR3 is possible. Preferred PD-L1 binding domains comprise a combination of HCDR1, HCDR2, and HCDR3 of SEQ ID NO: 54, SEQ ID NO: 55, and SEQ ID NO: 56; SEQ ID NO: 54, SEQ ID NO: 55, and SEQ ID NO: 58; SEQ ID NO: 60, SEQ ID NO: 3, and SEQ ID NO: 61; SEQ ID NO: 60, SEQ ID NO: 63, and SEQ ID NO: 56; SEQ ID NO: 65, SEQ ID NO: 66, and SEQ ID NO: 56; SEQ ID NO: 68, SEQ ID NO: 55, and SEQ ID NO: 56; SEQ ID NO: 70, SEQ ID NO: 71, and SEQ ID NO: 72; SEQ ID NO: 74, SEQ ID NO: 75, and SEQ ID NO: 76; SEQ ID NO: 78, SEQ ID NO: 79, and SEQ ID NO: 80; SEQ ID NO: 82, SEQ ID NO: 83, and SEQ ID NO: 84; SEQ ID NO: 86, SEQ ID NO: 87, and SEQ ID NO: 88; SEQ ID NO: 90, SEQ ID NO: 79, and SEQ ID NO: 91; SEQ ID NO: 93, SEQ ID NO: 94, and SEQ ID NO: 95; SEQ ID NO: 68, SEQ ID NO: 55, and SEQ ID NO: 56; SEQ ID NO: 70, SEQ ID NO: 98, and SEQ ID NO: 99; SEQ ID NO: 93, SEQ ID NO: 101, and SEQ ID NO: 102; SEQ ID NO: 74, SEQ ID NO: 105, and SEQ ID NO: 106; or SEQ ID NO: 86, SEQ ID NO: 108, and SEQ ID NO: 88. The PD-L1 binding domain of such multispecific antibody may comprise a heavy chain variable region having any one of SEQ ID NO: 53, 57, 59, 62, 64, 67, 69, 73, 77, 81, 85, 89, 92, 96, 97, 100, 103, 104, or 107, or having at least 80%, 85%, 90%, 95%, 99%, preferably 95%, sequence identity to the framework regions thereof. In certain embodiments, the PD-L1 binding domain of such multispecific antibody also comprises a CH1 domain. Any CH1 domain may be used. An example of a suitable CHI domain is provided by the amino acid sequence provided as SEQ ID NO: 116.

In certain embodiments, the multispecific antibody may comprise any combination of the CD137 and PD-L1 binding domains as disclosed herein, see for instance Table 1. One such multispecific antibody is MCLA-145.

In certain embodiments, the multispecific antibody may further comprise any light chain. An example of a suitable light chain comprises a light chain variable region comprising a light chain CDR3 (LCDR3) with an amino acid sequence as set forth in SEQ ID NO:113, allowing for one, two, or three amino acid substitutions therein. In certain embodiments, the light chain variable region comprises a light chain CDR3 (LCDR3) with an amino acid sequence as set forth in SEQ ID NO:113. The light chain variable region may further comprise a light chain CDR1 (LCDR1) with an amino acid sequence as set forth in SEQ ID NO: 111, allowing for one, two, or three amino acid substitutions therein, and/or a light chain CDR2 (LCDR2) with an amino acid sequence as set forth in SEQ ID NO:112, allowing for one, two, or three amino acid substitutions therein. In certain embodiments, the light chain variable region comprises a light chain CDR1 (LCDR1) with an amino acid sequence as set forth in SEQ ID NO: 111; and/or a light chain CDR2 (LCDR2) with an amino acid sequence as set forth in SEQ ID NO:112. The light chain variable region of the multispecific antibody may comprise a light chain variable region having SEQ ID NO: 110, or having at least 80%, 85%, 90%, 95%, 99% sequence identity to the framework regions thereof. In certain embodiments, the light chain of such multispecific antibody also comprises a CL domain. Any CL domain may be used. An example of a suitable CL domain is provided by the amino acid sequence provided as SEQ ID NO: 115.

In certain embodiments, the multispecific antibody may further comprise an Fc region, or part thereof. Such Fc region may comprise any modification known in the art, such as for instance, but not limited to, modifications to eliminate or reduce Fc effector function, and/or modifications that promote heterodimerization of the different CH3 domains. Any Fc region may be used. An example of a suitable Fc region is provided by the amino acid sequences provided as SEQ ID NO: 116-120.

TABLE 1
Antibodies comprising combinations of heavy chain variable regions specific for CD137 and heavy chain variable
regions specific for PD-L1. Each of PB1-PB252 can be combined with the light chain disclosed herein.

| | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
|---|---|---|---|---|---|---|---|---|---|---|---|---|---|---|
| | NO: 1 | NO: 5 | NO: 9 | NO: 13 | NO: 17 | NO: 20 | NO: 24 | NO: 28 | NO: 31 | NO: 35 | NO: 39 | NO: 43 | NO: 46 | NO: 49 |
| SEQ ID | PB1 | PB2 | PB3 | PB4 | PB5 | PB6 | PB7 | PB8 | PB9 | PB10 | PB11 | PB12 | PB13 | PB14 |
| NO: 53 | | | | | | | | | | | | | | |
| SEQ ID | PB15 | PB16 | PB17 | PB18 | PB19 | PB20 | PB21 | PB22 | PB23 | PB24 | PB25 | PB26 | PB27 | PB28 |
| NO: 57 | | | | | | | | | | | | | | |
| SEQ ID | PB29 | PB30 | PB31 | PB32 | PB33 | PB34 | PB35 | PB36 | PB37 | PB38 | PB39 | PB40 | PB41 | PB42 |
| NO: 59 | | | | | | | | | | | | | | |
| SEQ ID | PB43 | PB44 | PB45 | PB46 | PB47 | PB48 | PB49 | PB50 | PB51 | PB52 | PB53 | PB54 | PB55 | PB56 |
| NO: 62 | | | | | | | | | | | | | | |
| SEQ ID | PB57 | PB58 | PB59 | PB60 | PB61 | PB62 | PB63 | PB64 | PB65 | PB66 | PB67 | PB68 | PB69 | PB70 |
| NO: 64 | | | | | | | | | | | | | | |
| SEQ ID | PB71 | PB72 | PB73 | PB74 | PB75 | PB76 | PB77 | PB78 | PB79 | PB80 | PB81 | PB82 | PB83 | PB84 |
| NO: 67 | | | | | | | | | | | | | | |
| SEQ ID | PB85 | PB86 | PB87 | PB88 | PB89 | PB90 | PB91 | PB92 | PB93 | PB94 | PB95 | PB96 | PB97 | PB98 |
| NO: 69 | | | | | | | | | | | | | | |
| SEQ ID | PB99 | PB100 | PB101 | PB102 | PB103 | PB104 | PB105 | PB106 | PB107 | PB108 | PB109 | PB110 | PB111 | PB112 |
| NO: 73 | | | | | | | | | | | | | | |
| SEQ ID | PB113 | PB114 | PB115 | PB116 | PB117 | PB118 | PB119 | PB120 | PB121 | PB122 | PB123 | PB124 | PB125 | PB126 |
| NO: 77 | | | | | | | | | | | | | | |
| SEQ ID | PB127 | PB128 | PB129 | PB130 | PB131 | PB132 | PB133 | PB134 | PB135 | PB136 | PB137 | PB138 | PB139 | PB140 |
| NO: 81 | | | | | | | | | | | | | | |
| SEQ ID | PB141 | PB142 | PB143 | PB144 | PB145 | PB146 | PB147 | PB148 | PB149 | PB150 | PB151 | PB152 | PB153 | PB154 |
| NO: 85 | | | | | | | | | | | | | | |
| SEQ ID | PB155 | PB156 | PB157 | PB158 | PB159 | PB160 | PB161 | PB162 | PB163 | PB164 | PB165 | PB166 | PB167 | PB168 |
| NO: 89 | | | | | | | | | | | | | | |
| SEQ ID | PB169 | PB170 | PB171 | PB172 | PB173 | PB174 | PB175 | PB176 | PB177 | PB178 | PB179 | PB180 | PB181 | PB182 |
| NO: 92 | | | | | | | | | | | | | | |
| SEQ ID | PB183 | PB184 | PB185 | PB186 | PB187 | PB188 | PB189 | PB190 | PB191 | PB192 | PB193 | PB194 | PB195 | PB196 |
| NO: 96 | | | | | | | | | | | | | | |
| SEQ ID | PB197 | PB198 | PB199 | PB200 | PB201 | PB202 | PB03 | PB204 | PB205 | PB206 | PB207 | PB208 | PB209 | PB210 |
| NO: 97 | | | | | | | | | | | | | | |
| SEQ ID | PB211 | PB212 | PB213 | PB214 | PB215 | PB216 | PB217 | PB218 | PB219 | PB220 | PB221 | PB222 | PB223 | PB224 |
| NO: 100 | | | | | | | | | | | | | | |
| SEQ ID | PB225 | PB226 | PB227 | PB228 | PB229 | PB230 | PB231 | PB232 | PB233 | PB234 | PB235 | PB236 | PB237 | PB238 |
| NO: 104 | | | | | | | | | | | | | | |
| SEQ ID | PB239 | PB240 | PB241 | PB242 | PB243 | PB244 | PB245 | PB246 | PB247 | PB248 | PB249 | PB250 | PB251 | PB252 |
| NO: 107 | | | | | | | | | | | | | | |

As used herein, “to comprise” and its conjugations is used in its non-limiting sense to mean that items following the word are included, but items not specifically mentioned are not excluded.

The articles “a” and “an” are used herein to refer to one or to more than one (i.e., to at least one) of the grammatical object of the article. By way of example, “an element” means one element or more than one element.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

The present disclosure is exemplified in the following examples. These examples do not limit the scope of the present disclosure.

### EXAMPLES

### Example 1—Preparation of Cell Pellets for VeraTag® Assays

T75 flasks were coated overnight with 2 Kg/mL anti-CD3 (clone OKT3, eBioscience, cat no 16-0037-85) in PBS. Next, Jurkat T cells expressing CD137 (Jurkat_CD137K) were added at a concentration of 1.8×10⁶ cells/mL in 50 mL medium (9% FBS-HI RPMI 1640, 2 mM L-Glutamine) and incubated for 4 hours at 37° C. The Jurkat cells were then co-cultured with CHO-K1 cells expressing PD-L1 (CHO-PD-L1) at a concentration of 0.45×10⁶ cells/mL in 50 mL medium (Jurkat to CHO). The cells were allowed to interact for 4 hours, then a bispecific antibody binding to CD137 and PD-L1, an anti-CD137 positive control antibody or a negative control antibody binding to RSV (10 mg/mL) was added for a further 2 hours. The cells were then collected from the flasks by resuspension and scraping and fixed as follows: following centrifugation for 10 min at 1,200 rpm (125×g), 4° C., the medium was poured off and the cell pellet loosened and resuspended in ice-cold PBS. Centrifugation was repeated twice whereby the PBS was poured off and the cell pellet was loosened by vortexing. After the second wash, the pellet was resuspended in 30 mL 10% neutral buffered formalin (10% NBF, catalog number 5701, Thermo Fisher Scientific) and rotated gently overnight at 4° C. After centrifugation for 10 min at 1,500 rpm, 4° C., the formalin was removed and the cell pellet resuspended in 80% ethanol at 25×10⁶ cells/mL and stored at 4° C. before processing as described previously (Shi et al, 2009).

Suitable bispecific antibodies binding to CD137 and PD-L1 are for instance those specifically disclosed herein.

### Example 2—PD-L1 Expression VeraTag® Assay

Cell pellets prepared in Example 1 were used for this assay. 4.5×10⁵ cells were placed on positively-charged glass slides (FisherScientific) and analyzed using the VeraTag® Technology as briefly described below.

Antigen retrieval was performed via heat using a pressure cooker (Biocare Medical). Following antigen retrieval, antibody pairs were added, and the DTT-mediated released fluorescent VeraTags were detected by capillary electrophoresis. The released VeraTags were normalized to sample buffer volume to give units of relative fluorescence per 4.5×10⁵ cells.

Antibodies used were anti-PD-L1 rabbit mAb E1L3N (Cell Signaling Technology cat #13684) and Pepsin digest of Goat Anti-Rabbit IgG(H+L) (Southern Biotech cat #4052-01) labeled with a fluorescent VeraTag® reporter via a disulfide bond. In the isotype control experiment, the PD-L1 antibody is replaced by rabbit IgG (Cell Signaling Technology cat #3900).

Results are shown in FIG. 9. The VeraTag® assay appears a suitable means for detecting the expression levels of PD-L1. Similar amounts of PD-L1 were measured in all three samples.

### Example 3—CD137 Expression VeraTag® Assay

Cell pellets prepared in Example 1 were used for this assay. 4.5×10⁵ cells were placed on positively-charged glass slides (FisherScientific) and analyzed using the VeraTag® Technology as briefly described below.

Antigen retrieval was performed via heat using a pressure cooker (Biocare Medical). Following antigen retrieval, antibody pairs were added, and the DTT-mediated released fluorescent VeraTags were detected by capillary electrophoresis. The released VeraTags were normalized to sample buffer volume to give units of relative fluorescence per 4.5×10⁵ cells.

Two different primary antibodies were evaluated: anti-CD137 mouse mAb M127 (BD Pharmingen cat #552532) and anti-CD137 mouse mAb BBK2 (ThermoFisher cat #MS-621). A goat anti-mouse IgG secondary antibody (Jackson ImmunoResearch cat #115-005-146) conjugated to VeraTag was paired with the primary antibody. In the isotype control experiment, the CD137 antibody is replaced by mouse IgG (BD Pharmingen cat #554121).

Results are shown in FIG. 10. The VeraTag® assay appears a suitable means for detecting the expression levels of CD137. Similar amounts of CD137 were measured in all three samples for both primary assay antibodies.

### Example 4—CD137 Clustering VeraTag® Assay

Cell pellets prepared in Example 1 were used for this assay. 4.5×10⁵ cells were placed on positively-charged glass slides (FisherScientific) and analyzed using the VeraTag® Technology, as briefly described below.

Antigen retrieval was performed via heat using a pressure cooker (Biocare Medical). Following antigen retrieval, antibody pairs were added, and the released fluorescent VeraTags were detected by capillary electrophoresis. The released VeraTags were normalized to sample buffer volume to give units of relative fluorescence per 4.5×10⁵ cells.

Two different primary antibodies were evaluated: anti-CD137 mouse mAb M127 (BD Pharmingen cat #552532) and anti-CD137 mouse mAb BBK2 (ThermoFisher cat #MS-621). Equal concentrations of anti-CD137 antibodies were labeled with either a fluorescent VeraTag reporter or biotin.

Results are shown in FIG. 11. The VeraTag® assay appears a suitable means for detecting the clustering of CD137. The VeraTag® signal appears stronger when the BBK antibody is used as primary assay antibody as compared to the M127 antibody.

### Example 5—CD137-PD-L1 Proximity VeraTag® Assay

Cell pellets prepared in Example 1 were used for this assay. 4.5×10⁵ cells were placed on positively-charged glass slides (FisherScientific) and analyzed using the VeraTag® Technology, as briefly described below.

Antigen retrieval was performed via heat using a pressure cooker (Biocare Medical). Following antigen retrieval, antibody pairs were added, and the released fluorescent VeraTags were detected by capillary electrophoresis. The released VeraTags were normalized to sample buffer volume to give units of relative fluorescence per 4.5×10⁵ cells.

CD137-PD-L1 proximity was measured by the proximity dependent release of a VeraTag reporter from anti-CD137 mouse mAb M127 (BD Pharmingen cat #552532, ectodomain) or mouse mAb BBK2 (ThermoFisher cat #MS-621, ectodomain) paired with anti-PD-L1 rabbit mAb E1L3N (Cell Signaling Technology cat #13684, c-terminus) and a biotinylated goat anti-rabbit IgG secondary antibody (Rockland Immunochemicals cat #611-101-122). In the isotype control experiment, the PD-L1 antibody was replaced by rabbit IgG (Cell Signaling Technology cat #3900). Results are shown in FIG. 12. The VeraTag® assay appears a suitable means for detecting a CD137-PD-L1 complex. Sample B shows the strongest VeraTag signal for both primary assay antibodies. This indicates that the CD137xPD-L1 bispecific antibody binds to CD137 and PD-L1 simultaneously and clusters these antigens, and thus the cells expressing these antigens.

SEQUENCES

SEQ ID NO: 1: Heavy Chain Variable region
QVQLVQSGSELKKPGASVKVSCKASGYTFTNFAMNWVRRAPGQGLEWMGWINTNTGNPT
YAQGFTGRFVFSLDTSVNTAYLQISSLKAEDTAVYYCARDWGVIGGHYMDVWGKGTTVTV
SS
SEQ ID NO: 2: HCDR1 according to KABAT from SEQ ID NO: 1
NFAMN
SEQ ID NO: 3: HCDR2 according to KABAT from SEQ ID NO: 1
WINTNTGNPTYAQGFTG
SEQ ID NO: 4: HCDR3 according to KABAT from SEQ ID NO: 1
DWGVIGGHYMDV
SEQ ID NO: 5: Heavy Chain Variable region
QVQLVQSGAEVKKPGASVKVSCKASGYTFTSYGISWVRQAPGQGLEWMGWISAYNGNTN
YAQKLQGRVTMTTDTSTSTAYMELRSLRSDDTAVYYCARDSDGYGPKAFDYWGQGTLVT
VSS
SEQ ID NO: 6: HCDR1 according to KABAT from SEQ ID NO: 5
SYGIS
SEQ ID NO: 7: HCDR2 according to KABAT from SEQ ID NO: 5
WISAYNGNTNYAQKLQG
SEQ ID NO: 8: HCDR3 according to KABAT from SEQ ID NO: 5
DSDGYGPKAFDY
SEQ ID NO: 9: Heavy Chain Variable region
EVQLVQSGAEVKKPGESLKISCKGSGYSFTSYWIGWVRQMPGKGLEWMGIIYPDDSDTRYS
PSFQGQVTISADKSSSTAYLQWSSLKASDTAMYYCASFYTGIVGATGAFDVWGQGTTVTVS
S
SEQ ID NO: 10: HCDR1 according to KABAT from SEQ ID NO: 9
SYWIG
SEQ ID NO: 11: HCDR2 according to KABAT from SEQ ID NO: 9
IIYPDDSDTRYSPSFQG
SEQ ID NO: 12: HCDR3 according to KABAT from SEQ ID NO: 9
FYTGIVGATGAFDV
SEQ ID NO: 13: Heavy Chain Variable region
QVQLVQSGAEVKKPGSSVKVSCKASGGTFSSDAISWVRQAPGQGLEWMGGMIPILGTANY
AQKFQGRVTITADRSTSTAYMELSSLRSEDTAVYYCVRGATYYYGSGTYYSINWFDPWGQ
GTLVTVSS
SEQ ID NO: 1 : HCDR1 according to KABAT from SEQ ID NO: 13
SDAIS
SEQ ID NO: 15: HCDR2 according to KABAT from SEQ ID NO: 13
GMIPILGTANYAQKFQG
SEQ ID NO: 16: HCDR3 according to KABAT from SEQ ID NO: 13
GATYYYGSGTYYSINWFDP
SEQ ID NO: 17: Heavy Chain Variable region
QVQLVQSGSELKKPGASVKVSCRASGYTFTNFAMTWVRQAPGQGPEYMGWINTNTGNPTY
AQGFTGRFVFSLDTSVNTAYLQISSLKAEDTAVYYCARDWASVMVRGDLDYWGQGTLVTV
SS
SEQ ID NO: 18: HCDR1 according to KABAT from SEQ ID NO: 17
NFAMT
SEQ ID NO: 19: HCDR3 according to KABAT from SEQ ID NO: 17
DWASVMVRGDLDY
SEQ ID NO: 20: Heavy Chain Variable region
QVQLVQSGAEVKKPGASVKVSCKVSGYTLSELSIHWVRQAPGKGVEWMGGFYPEDVEPIY
ARKFQGRVTMTEDTSTDTAYMELNSLRSEDTAVYYCAAEGFDNYGSGIRGNWFDPWGQGT
LVTVSS
SEQ ID NO: 21: HCDR1 according to KABAT from SEQ ID NO: 20
ELSIH
SEQ ID NO: 22: HCDR2 according to KABAT from SEQ ID NO: 20
GFYPEDVEPIYARKFQG
SEQ ID NO: 23: HCDR3 according to KABAT from SEQ ID NO: 20
EGFDNYGSGIRGNWFDP
SEQ ID NO: 24: Heavy Chain Variable region
EVQLVQSGAEVKKPGASVKVSCKVSGYTLTELSMHWVRQSPGKGLEWMGSFYPEDGETIY
AQKFQGRITMTEDTSADTAYMELSSLRSEDTAVYYCATEGVGVIRGNWFDPWGQGTLVTV
SS
SEQ ID NO: 25: HCDR1 according to KABAT from SEQ ID NO: 24
ELSMH
SEQ ID NO: 26: HCDR2 according to KABAT from SEQ ID NO: 24
SFYPEDGETIYAQKFQG
SEQ ID NO: 27: HCDR3 according to KABAT from SEQ ID NO: 24
EGVGVIRGNWFDP
SEQ ID NO: 28: Heavy Chain Variable region
EVQLVQSGAEVKKPGESLKISCKGSGYSFTSYWIGWVRQMPGKGLEWMGIIFPDDSDTRYS
PSFQGQVTISADKSISTAYLQWSSLKPSDTAMYYCVRLGGYSGYAEDFVDFWGQGTLVTVS
S
SEQ ID NO: 29: HCDR2 according to KABAT from SEQ ID NO: 28
IIFPDDSDTRYSPSFQG
SEQ ID NO: 30: HCDR3 according to KABAT from SEQ ID NO: 28
LGGYSGYAEDFVDF
SEQ ID NO: 31: Heavy Chain Variable region
EVQLVQSGAEVKKPGASVKVSCKVSGYTLTKLSMHWVRQAPGKGLEWMGGFEPEDGETIN
AQKFQGRVTMTEDTSTDTAYMELSSLRSEDTAVYYCATDLRLGASYYYSYMDVWGRGTM
VTVSS
SEQ ID NO: 32: HCDR1 according to KABAT from SEQ ID NO: 31
KLSMH
SEQ ID NO: 33: HCDR2 according to KABAT from SEQ ID NO: 31
GFEPEDGETINAQKFQG
SEQ ID NO: 34: HCDR3 according to KABAT from SEQ ID NO: 31
DLRLGASYYYSYMDV
SEQ ID NO: 35: Heavy Chain Variable region
QITLKESGPTLVKPTQTLTLSCTFSGFSLSTSGMSVGWIRQPPGKALEWLALIYWNDDKYFSP
SLKSRLTITKDTSKNQVVLTLTNMDPVDTATYYCAHTLWGSDDVFDVWGQGTMVTVSS
SEQ ID NO: 36: HCDR1 according to KABAT from SEQ ID NO: 35
TSGMSVG
SEQ ID NO: 37: HCDR2 according to KABAT from SEQ ID NO: 35
LIYWNDDKYFSPSLKS
SEQ ID NO: 38: HCDR3 according to KABAT from SEQ ID NO: 35
TLWGSDDVFDV
SEQ ID NO: 39: Heavy Chain Variable region
EVQLVQSGAEVKKPGESLKISCKVSGYSFTNYWIGWVRQMPGKGLEWMGIIYPGDSDTRYS
PSFQGQVTISADKSISTAYLQWHTLKASDTAMYYCARHQGYSFSGSHIDDYWGQGTLVTVS
S
SEQ ID NO: 40: HCDR1 according to KABAT from SEQ ID NO: 39
NYWIG
SEQ ID NO: 41: HCDR2 according to KABAT from SEQ ID NO: 39
IIYPGDSDTRYSPSFQG
SEQ ID NO: 42: HCDR3 according to KABAT from SEQ ID NO: 39
HQGYSFSGSHIDDY
SEQ ID NO: 43: Heavy Chain Variable region
EVQLVQSGAEVRKPGESLKISCKGSGYSFTTYWIGWVRQMPGKGLEWMGIIYPGDSDTRYS
PSFQGQVTISADKSISTVYLQWSSLKASDTAMYYCARHAGFIITSQNIDDYWGQGTLVTVSS
SEQ ID NO: 44: HCDR1 according to KABAT from SEQ ID NO: 43
TYWIG
SEQ ID NO: 45 : HCDR3 according to KABAT from SEQ ID NO: 43
HAGFIITSQNIDDY
SEQ ID NO: 46: Heavy Chain Variable region
EVQLVQSGSELKKPGASVKVSCKASGYTFTNFAMNWVRQAPGQGLEWMGWINTNTGNPT
YAQDFTGRFVFSLDTSGNTAYLQISSLKAEDTAVYYCARDWGLVAIGYFDYWGQGTLVTV
SS
SEQ ID NO: 47: HCDR2 according to KABAT from SEQ ID NO: 46
WINTNTGNPTYAQDFTG
SEQ ID NO: 48: HCDR3 according to KABAT from SEQ ID NO: 46
DWGLVAIGYFDY
SEQ ID NO: 49: Heavy Chain Variable region
QITLKESGPTLVKPTQTLTLTCTFSGFSLSTTGVGVNWIRQPPGEALEWLALIYWNDDTYYSP
SLKSRLTITKDTSKNQVVLTMTNMDPVDTATYYCAHEGIIGFLGGNWFDPWGQGTLVTVSS
SEQ ID NO: 50: HCDR1 according to KABAT from SEQ ID NO: 49
TTGVGVN
SEQ ID NO: 51: HCDR2 according to KABAT from SEQ ID NO: 49
LIYWNDDTYYSPSLKS
SEQ ID NO: 52: HCDR3 according to KABAT from SEQ ID NO: 49
EGIIGFLGGNWFDP
SEQ ID NO: 53: Heavy Chain Variable region
QVQLVQSGSELKKPGASVKVSCKASGYTFTSHAMNWVRQAPGQGLEWMGWINPNTGNPT
YAQGFTGRFVFSLDTSVSTAYLQISSLKAEDTAVYYCARDRKYVTNWVFAEDFQHWGQGT
LVTVSS
SEQ ID NO: 54: HCDR1 according to KABAT from SEQ ID NO: 53
SHAMN
SEQ ID NO: 55: HCDR2 according to KABAT from SEQ ID NO: 53
WINPNTGNPTYAQGFTG
SEQ ID NO: 56: HCDR3 according to KABAT from SEQ ID NO: 53
DRKYVTNWVFAEDFQH
SEQ ID NO: 57: Heavy Chain Variable region
QVQLVQSGSELKKPGASVKVSCKASGYTFTSHAMNWVRQAPGQGLEWMGWINPNTGNPT
YAQGFTGRFVFSLDTSVSTAYLQISSLKAEDTAVYYCAIDRGYMSNWVFAEYFPHWGQGTL
VTVSS
SEQ ID NO: 58: HCDR3 according to KABAT from SEQ ID NO: 57
DRGYMSNWVFAEYFPH
SEQ ID NO: 59: Heavy Chain Variable region
QVQLVQSGSELKKPGASVKVSCKASGYTFTSYAMNWVRQAPGQGLEWMGWINTNTGNPT
YAQGFTGRFVFSLDTSVSTAYLQISSLKAEDTAVYYCATDRGYISSWVFAEDFQHWGQGTL
VTVSS
SEQ ID NO: 60: HCDR1 according to KABAT from SEQ ID NO: 59
SYAMN
SEQ ID NO: 61: HCDR3 according to KABAT from SEQ ID NO: 59
DRGYISSWVFAEDFQH
SEQ ID NO: 62: Heavy Chain Variable region
QVQLVQSGSELKKPGASVKVSCTASGYTFTSYAMNWVRQAPGQRLEWMACVNPNTGSPT
YAQGSTGRFVVSLDTSVSTAYLQISSLKAEDTAVYYCARDRKYVTNWVFAEDFQHWGHGT
LVTVSS
SEQ ID NO: 63: HCDR2 according to KABAT from SEQ ID NO: 62
CVNPNTGSPTYAQGSTG
SEQ ID NO: 64: Heavy Chain Variable region
QVQLVQSGSELKKPGASVKVSCKASGYTFTNYAMNWVRQAPGQGLEWMGWMNPNTGNP
TYAQGSTGRFVVSLDTSVSTAYLQISSLKAEDTAVYYCARDRKYVTNWVFAEDFQHWGRG
TLVTVSS
SEQ ID NO: 65: HCDR1 according to KABAT from SEQ ID NO: 64
NYAMN
SEQ ID NO: 66: HCDR2 according to KABAT from SEQ ID NO: 64
WMNPNTGNPTYAQGSTG
SEQ ID NO: 67: Heavy Chain Variable region
QVQLVQSGSELKKPGASVKVSCKASGYTFTNYAINWVRQAPGQGLEWMGWINPNTGNPTY
AQGFTGRFVFSLDTSVSTAYLQISSLKAEDTAVYYCARDRKYVTNWVFAEDFQHWGRGTL
VTVSS
SEQ ID NO: 68: HCDR1 according to KABAT from SEQ ID NO: 67
NYAIN
SEQ ID NO: 69: Heavy Chain Variable region
EVQLVQSGAEVKKPGSSVKVSCKASGDTFNTYSITWVRQAPGQGLEWMGSIVPIFGTINNA
QKFQGRVTITADKSANTAYMELSSLRSEDTAVYYCARDNTMVRGVDYYYMDVWGKGTM
VTVSS
SEQ ID NO: 70: HCDR1 according to KABAT from SEQ ID NO: 69
TYSIT
SEQ ID NO: 71: HCDR2 according to KABAT from SEQ ID NO: 69
SIVPIFGTINNAQKFQG
SEQ ID NO: 72: HCDR3 according to KABAT from SEQ ID NO: 69
DNTMVRGVDYYYMDV
SEQ ID NO: 73: Heavy Chain Variable region
EVQLVQSGAEVKKPGSSVKVSCKASGGIFSTYAISWVRQAPGQGLEWMGGIIPIFDTPNYAQ
KFQGRVTITADKSTSTAYMDLSSLRSEDTAVYYCAKNVRGYSAYDLDYWGQGTLVTVSS
SEQ ID NO: 74: HCDR1 according to KABAT from SEQ ID NO: 73
TYAIS
SEQ ID NO: 75: HCDR2 according to KABAT from SEQ ID NO: 73
GIIPIFDTPNYAQKFQG
SEQ ID NO: 76: HCDR3 according to KABAT from SEQ ID NO: 73
NVRGYSAYDLDY
SEQ ID NO: 77: Heavy Chain Variable region
EVQLVQSGAEVKNPGSSVKVSCKATGGTFNTYGTNWVRQAPGQGLEWMGGIIPIFGTANY
AQKFQGRVTITADKSTTTAYMEVSSLRSEDTAVYYCARGGADMGTLDYWGQGTLVTVSS
SEQ ID NO: 78: HCDR1 according to KABAT from SEQ ID NO: 77
TYGTN
SEQ ID NO: 79: HCDR2 according to KABAT from SEQ ID NO: 77
GIIPIFGTANYAQKFQG
SEQ ID NO: 80: HCDR3 according to KABAT from SEQ ID NO: 77
GGADMGTLDY
SEQ ID NO: 81: Heavy Chain Variable region
EVQLVQSGAEVMRPGSSVKVSCKASGGIFNTYTIIWVRQAPGQGLEWMGGIIPIFDTPNFAQ
KFQGRLTITADKSTNTAYMELTSLRSEDTAVYYCAREGCNHGVCYPYWGQGTLVTVSS
SEQ ID NO: 82: HCDR1 according to KABAT from SEQ ID NO: 81
TYTII
SEQ ID NO: 83: HCDR2 according to KABAT from SEQ ID NO: 81
GIIPIFDTPNFAQKFQG
SEQ ID NO: 84: HCDR3 according to KABAT from SEQ ID NO: 81
EGCNHGVCYPY
SEQ ID NO: 85: Heavy Chain Variable region
QVQLVQSGAEVKKPGSSVKVSCKASGDTFRSYGITWVRQAPGQGLEWMGGIIPIFGTTNYA
QKFQGRVTITADKSTSTVYMELSSLRSEDTAVYYCARRRGYSNPHWLDPWGQGTLVTVSS
SEQ ID NO: 86: HCDR1 according to KABAT from SEQ ID NO: 85
SYGIT
SEQ ID NO: 87: HCDR2 according to KABAT from SEQ ID NO: 85
GIIPIFGTTNYAQKFQG
SEQ ID NO: 88: HCDR3 according to KABAT from SEQ ID NO: 85
RRGYSNPHWLDP
SEQ ID NO: 89: Heavy Chain Variable region
QVQLVQSGAEVKKPGSSVKVSCKASGGTFSTYGILWVRQAPGQGLEWMGGIIPIFGTANYA
QKFQGRVTITADISTSTAYMELSSLRSEDTAVYYCARGGGNYYEFVYWGQGTLVTVSS
SEQ ID NO: 90: HCDR1 according to KABAT from SEQ ID NO: 89
TYGIL
SEQ ID NO: 91: HCDR3 according to KABAT from SEQ ID NO: 89
GGGNYYEFVY
SEQ ID NO: 92: Heavy Chain Variable region
EVQLVQSGAEVKKPGSSVRVSCKASGGTFNTYAINWVRQAPGQGLEWVGRIIPIFDTANYA
QKFQGRVTISADKSTTTAYMELSSLRSEDTAVFYCAKDETGYSSSNFQHWGQGTLVTVSS
SEQ ID NO: 93: HCDR1 according to KABAT from SEQ ID NO: 92
TYAIN
SEQ ID NO: 94: HCDR2 according to KABAT from SEQ ID NO: 92
RIIPIFDTANYAQKFQG
SEQ ID NO: 95: HCDR3 according to KABAT from SEQ ID NO: 92
DETGYSSSNFQH
SEQ ID NO: 96: Heavy Chain Variable region
QVQLVQSGSELKKPGASVKVSCKASGYTFTNYAINWVRQAPGQGLEWMGWINPNTGNPTY
AQGFTGRFVFSLDTSVSTAYLQISSLKAEDTAVYYCARDRKYVTNWVFAEDFQHWGQGTL
VTVSS
SEQ ID NO: 97: Heavy Chain Variable region
QVQLVQSGAEVKRPGSSVKVSCKASGGTFNTYSITWVRQAPGQGLEWMGGIIPVFGTSKYA
QKFQDRVTITADKSTNTAYMELSSLRSEDTAVYYCARDPSFSSSSGWFDPWGQGTLVTVSS
SEQ ID NO: 98: HCDR2 according to KABAT from SEQ ID NO: 97
GIIPVFGTSKYAQKFQD
SEQ ID NO: 99: HCDR3 according to KABAT from SEQ ID NO: 97
DPSFSSSSGWFDP
SEQ ID NO: 100: Heavy Chain Variable region
QVQLVQSGAEVKKPGSSVKVSCKASGGTFNTYAINWVRQAPGQGLEWMGGIIPIFDTANYA
QRFQGRVTITADKSTSTAYMELSSLRSEDTAVYFCAKDQTGYSSTLFDYWGQGTLVTVSS
SEQ ID NO: 101: HCDR2 according to KABAT from SEQ ID NO: 100
GIIPIFDTANYAQRFQG
SEQ ID NO: 102: HCDR3 according to KABAT from SEQ ID NO: 100
DQTGYSSTLFDY
SEQ ID NO: 103: Heavy Chain Variable region
QVQLVQSGSELKKPGASVKVSCKASGYTFTSHAMNWVRQAPGQGLEWMGWINPNTGNPT
YAQGFTGRFVFSLDTSVSTAYLQISSLKAEDTAVYYCAIDRGYMSNWVFAEYFPHWGQGTL
VTVSS
SEQ ID NO: 104: Heavy Chain Variable region
EVQLVQSGAEVKKPGSSVKVSCKASGGTFSTYAISWVRQAPGQGLEWMGWIIPIFDTGNYA
QKIQGRVTITADKSTSTAYMELTSLRSEDTAVYYCARHDYTNTVDAFDIWGQGTMVTVSS
SEQ ID NO: 105: HCDR2 according to KABAT from SEQ ID NO: 104
WIIPIFDTGNYAQKIQG
SEQ ID NO: 106: HCDR3 according to KABAT from SEQ ID NO: 104
HDYTNTVDAFDI
SEQ ID NO: 107: Heavy Chain Variable region
QVQLVQSGAEVKKPGSSVKVSCKASGDTFRSYGITWVRQAPGQGLEWMGGIIPVFGTTNYA
QKFQGRVTITADKSTSTVFMELNSLRSEDTAVYYCARRRGYSNPHWLDPWGQGTLVTVSS
SEQ ID NO: 108: HCDR2 according to KABAT from SEQ ID NO: 107
GIIPVFGTTNYAQKFQG
SEQ ID NO: 109: Amino acid sequence of human common light chain
IGKV1-39/jk1
DIQMTQSPSSLSASVGDRVTITCRASQSISSYLNWYQQKPGKAPKLLIYAASSLQSGVPSRFS
GSGSGTDFTLTISSLQPEDFATYYCQQSYSTPPTFGQGTKVEIK
RTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 110: Amino acid sequence of common light chain variable domain
DIQMTQSPSSLSASVGDRVTITCRASQSISSYLNWYQQKPGKAPKLLIYAASSLQSGVPSRFS
GSGSGTDFTLTISSLQPEDFATYYCQQSYSTPPTFGQGTKVEIK
SEQ ID NO: 111: LCDR1 according to KABAT from SEQ ID NO: 110
RASQSISSYLN
SEQ ID NO: 112: LCDR2 according to KABAT from SEQ ID NO: 110
AASSLQS
SEQ ID NO: 113: LCDR3 according to KABAT from SEQ ID NO: 110
QQSYSTPPT
SEQ ID NO: 114: Amino acid sequence of common light chain constant domain
RTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 115: Amino acid sequence of light chain constant domain
RTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 116: Amino acid sequence of CH1
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRV
SEQ ID NO: 117: Amino acid sequence of the hinge
EPKSCDKTHTCPPCP
SEQ ID NO: 118: Amino acid sequence of CH2
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
SEQ ID NO: 119: Amino acid sequence of CH3 with KK mutations
GQPREPQVYTKPPSREEMTKNQVSLKCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 120: Amino acid sequence of CH3 with DE mutations
GQPREPQVYTDPPSREEMTKNQVSLTCEVKGFYPSDIAVEWESNGQPENNYKTTPPVLDSD
GSFFLYSKLTVDKSRWQQGNVFSCSVMHEALHNHYTQKSLSLSPGK

1-47. (canceled)

48. A method for detecting and/or quantifying the presence in a sample of clustering of at least two cell surface moieties, comprising a first cell surface moiety and a second cell surface moiety, wherein the first and second cell surface moieties are expressed by different cells or different types of cells, the method comprising:
contacting a sample in which the first and second cell surface moieties have been exposed to an agent having binding specificity for at least the first and second cell surface moieties with a first binding molecule that specifically binds to the first cell surface moiety and a second binding molecule that specifically binds to the second cell surface moiety, wherein at least one of the first binding molecule and the second binding molecule comprises a molecular tag which is not detected unless the first and second cell surface moieties are in proximity of each other; and
detecting the presence or absence and/or measuring the amount of the molecular tag to detect and/or quantify the presence in the sample of clustering of the first cell surface moiety and the second cell surface moiety.

49. The method according to claim 48, wherein the method comprises:
a) contacting the sample with a first binding molecule that specifically binds to the first cell surface moiety and a second binding molecule that specifically binds to the second cell surface moiety,
wherein the first binding molecule comprises a molecular tag attached thereto via a cleavable linker and the second binding molecule comprises a cleavage inducing moiety; or
b) contacting the sample with a first binding molecule that specifically binds to the first cell surface moiety and a second binding molecule that specifically binds to the second cell surface moiety,
wherein the second binding molecule comprises a molecular tag attached thereto via a cleavable linker and the first binding molecule comprises a cleavage inducing moiety;
inducing cleavage of the molecular tag; and
detecting the presence or absence, or measuring the amount, of released molecular tag thereby detecting or quantifying clustering of the first cell surface moiety with the second cell surface moiety in the sample.

50. The method according to claim 48, wherein the method comprises:
a) contacting the sample with a first binding molecule that specifically binds to the first cell surface moiety, a second binding molecule that specifically binds to the second cell surface moiety, and a third binding molecule,
wherein the first binding molecule comprises a molecular tag attached thereto via a cleavable linker and the third binding molecule binds to the second binding molecule and comprises a cleavage inducing moiety; or
b) contacting the sample with a first binding molecule that specifically binds to the first cell surface moiety, a second binding molecule that specifically binds to the second cell surface moiety, and a third binding molecule,
wherein the second binding molecule comprises a molecular tag attached thereto via a cleavable linker and the third binding molecule binds to the first binding molecule and comprises a cleavage inducing moiety; or
c) contacting the sample with a first binding molecule that specifically binds to the first cell surface moiety, a second binding molecule that specifically binds to the second cell surface moiety, and a third binding molecule,
wherein the first binding molecule comprises a cleavage inducing moiety and the third binding molecule binds to the second binding molecule and comprises a molecular tag attached thereto via a cleavable linker; or
d) contacting the sample with a first binding molecule that specifically binds to the first cell surface moiety, a second binding molecule that specifically binds to the second cell surface moiety, and a third binding molecule,
wherein the second binding molecule comprises a cleavage inducing moiety and the third binding molecule binds to the first binding molecule and comprises a molecular tag attached thereto via a cleavable linker;
inducing cleavage of the molecular tag; and
detecting the presence or absence, or measuring the amount, of released molecular tag thereby detecting or quantifying clustering of the first cell surface moiety with the second cell surface moiety in the sample.

51. The method according to claim 48, wherein the method comprises:
a) contacting the sample with a first binding molecule that specifically binds to the first cell surface moiety, a second binding molecule that specifically binds to the second cell surface moiety, a third binding molecule, and a fourth binding molecule,
wherein the third binding molecule binds to the first binding molecule and comprises a molecular tag attached thereto via a cleavable linker, and the fourth binding molecule binds to the second binding molecule and comprises a cleavage inducing moiety; or
b) contacting the sample with a first binding molecule that specifically binds to the first cell surface moiety, a second binding molecule that specifically binds to the second cell surface moiety, a third binding molecule, and a fourth binding molecule,
wherein the third binding molecule binds to the first binding molecule and comprises a cleavage inducing moiety; and the fourth binding molecule binds to the second binding molecule and comprises a molecular tag attached thereto via a cleavable linker,
inducing cleavage of the molecular tag; and
detecting the presence of absence, or measuring the amount, of released molecular tag thereby detecting or quantifying clustering of the first cell surface moiety with the second cell surface moiety in the sample.

52. The method according to claim 48, wherein the sample is a tissue sample, a blood sample, a tumor biopsy, or cultured cells.

53. A method for detecting and/or quantifying expression in a sample of at least two cell surface moieties, comprising a first cell surface moiety and a second cell surface moiety, wherein the first and second cell surface moieties are expressed by different cells or different types of cells, the method comprising:
contacting a tumor biopsy sample from a subject having cancer with at least one binding molecule that detects a first cell surface moiety and at least one binding molecule that detects a second cell surface moiety,
wherein at least one binding molecule that detects the first cell surface moiety and at least one binding molecule that detects the second cell surface moiety comprise a molecular tag; and
detecting and/or quantifying the presence or absence of the molecular tags to detect the expression of the first cell surface moiety and of the second cell surface moiety in the sample.

54. The method according to claim 48, wherein the first cell surface moiety is expressed by an immune effector cell, and the second cell surface moiety is expressed by a tumor cell or an immune cell.

55. The method according to claim 48, wherein at least one cell surface moiety is CD137 or another immune effector cell co-stimulatory moiety, and/or at least one cell surface moiety is PD-L1 or another tumor-associated moiety or immune checkpoint moiety.

56. The method according to claim 48, wherein the agent having binding specificity for the cell surface moieties is a multispecific antibody.

57. The method according to claim 56, wherein the multispecific antibody is a bispecific antibody that binds to CD137 or another immune effector cell co-stimulatory molecule and PD-L1 or another tumor-associated moiety or immune checkpoint moiety.

58. The method according to claim 57, wherein the binding domain of the bispecific antibody that binds to CD137 comprises a heavy chain variable region having a heavy chain CDR1 (HCDR1) with an amino acid sequence as set forth in any one of SEQ ID NO: 2, 6, 10, 14, 18, 21, 25, 32, 36, 40, 44, or 50; and a heavy chain CDR2 (HCDR2) with an amino acid sequence as set forth in any one of SEQ ID NO: 3, 7, 11, 15, 22, 26, 29, 33, 37, 41, 47, or 51; and a heavy chain CDR3 (HCDR3) with an amino acid sequence as set forth in any one of SEQ ID NO: 4, 8, 12, 16, 19, 23, 27, 30, 34, 38, 42, 45, 48, or 52.

59. The method according to claim 57, wherein the binding domain of the bispecific antibody that binds to CD137 comprises a heavy chain variable region having any one of SEQ ID NO: 1, 5, 9, 13, 17, 20, 24, 28, 31, 35, 39, 43, 46, or 49, or having at least 80%, 85%, 90%, 95%, 99% sequence identity thereto.

60. The method according to claim 57, wherein the binding domain of the bispecific antibody that binds to PD-L1 comprises a heavy chain CDR1 (HCDR1) with an amino acid sequence as set forth in any one of SEQ ID NO: 54, 60, 65, 68, 70, 74, 78, 82, 86, 90, or 93; and a heavy chain CDR2 (HCDR2) with an amino acid sequence as set forth in any one of SEQ ID NO: 55, 3, 63, 66, 71, 75, 79, 83, 87, 94, 98, 101, 105, or 108; and a heavy chain variable region having a heavy chain CDR3 (HCDR3) with an amino acid sequence as set forth in any one of SEQ ID NO: 56, 58, 61, 72, 76, 80, 84, 88, 91, 95, 99, 102, or 106.

61. The method according to claim 57, wherein the binding domain of the bispecific antibody that binds to PD-L1 comprises a heavy chain variable region having any one of SEQ ID NO: 53, 57, 59, 62, 64, 67, 69, 73, 77, 81, 85, 89, 92, 96, 97, 100, 103, 104, or 107, or having at least 80%, 85%, 90%, 95%, 99% sequence identity thereto.

62. A method for predicting the responsiveness of a subject, in particular a cancer patient, to an agent or agents binding a first cell surface moiety and a second cell surface moiety, wherein the first and second cell surface moieties are expressed by different cells or different types of cells, in particular a moiety expressed on an immune effector cell and a moiety expressed on a tumor cell or immune cell, the method comprising:
detecting the expression levels of a first cell surface moiety and a second cell surface member in a biological sample from a subject;
determining whether the expression levels of the first cell surface moiety and the second cell surface moiety in the subject's sample is above or below a threshold level; and
predicting that the subject is likely to respond to an agent or agents binding the first cell surface moiety and the second cell surface moiety if the expression levels of the first cell surface moiety and the second cell surface moiety in the subject's sample is equal to or above the threshold level.

63. The method according to claim 62, wherein the expression levels of a first cell surface moiety and a second cell surface member are measured by
contacting a tumor biopsy sample from a subject having cancer with at least one binding molecule that detects a first cell surface moiety and at least one binding molecule that detects a second cell surface moiety,
wherein at least one binding molecule that detects the first cell surface moiety and at least one binding molecule that detects the second cell surface moiety comprise a molecular tag; and
detecting and/or quantifying the presence or absence of the molecular tags to detect the expression of the first cell surface moiety and of the second cell surface moiety in the sample.

64. A method for treating a subject in need thereof, in particular a subject having cancer, the method comprising:
predicting responsiveness of a subject to an agent or agents binding a first cell surface moiety and a second cell face moiety using the method according to claim 62; and
administering an agent or agents binding the first cell surface moiety and the second cell surface to a subject that is likely to respond.

65. The method according to claim 62, wherein at least one cell surface moiety is CD137 or another immune effector cell co-stimulatory moiety and/or at least one cell surface moiety is PD-L1 or another tumor-associated moiety or immune checkpoint moiety

66. The method according to claim 62, wherein the agent binding the cell surface moieties is a multispecific antibody, such as a bispecific or trispecific antibody, in particular a bispecific antibody that specifically binds to CD137 or another immune effector cell co-stimulatory moiety and PD-L1 or another tumor-associated moiety or immune checkpoint moiety.

67. A method for determining the effectiveness of an agent, the agent comprising at least a binding domain that specifically binds to a first cell surface moiety and a binding domain that specifically binds to a second cell surface moiety, the method comprising detecting and/or quantifying clustering of a first cell surface moiety with a second cell surface moiety in a biological sample of a subject under treatment with the agent by using the method according to claim 48.

68. A method for confirming the mode of action of an agent, the agent comprising at least a binding domain that specifically binds to a first cell surface moiety and a binding domain that specifically binds to a second cell surface moiety, the method comprising detecting and/or quantifying clustering of a first cell surface moiety with a second cell surface moiety in a biological sample of a subject under treatment with the agent by using the method according to claim 48.

69. The method according to claim 20, wherein the mode of action is the simultaneous binding of the agent to the first and second cell surface moiety.

70. The method according to claim 68, wherein the mode of action is clustering of two or more cell surface moieties, in particular wherein the clustering is of two or more cell surface moieties expressed on an immune effector cell, in particular the clustering of two or more CD137 proteins.

71. A method for treating a subject in need thereof, in particular a subject having cancer, the method comprising:
treating a subject with an agent binding a first cell surface moiety and a second cell surface moiety;
analyzing the effectiveness of the agent or the mode of action of the agent using the method according to claim 67; and
continuing or adapting the treatment based on the outcome of the analysis or confirmation.

72. A method for screening one or more test agents for the ability to induce clustering of at least a first cell surface moiety with a second cell surface moiety, the method comprising:
contacting one or more test cell cultures with a test agent,
wherein the test cell culture comprises a cell expressing a first cell surface moiety, and a cell expressing a second cell surface moiety;
detecting or quantifying the level of clustering of the first and second cell surface moieties using a method according to claim 48; and
comparing the level of clustering with the level of clustering detected for the clustering in a control cell culture not contacted with the test agent or contacted with a reference agent,
wherein the control cell culture comprises the first cell surface moiety, and the second cell surface moiety.

73. The method according to claim 72, further comprising selecting a test agent that induces an equal or higher level of clustering than the level of clustering in the control cell culture.

74. The method according to claim 72, wherein at least one cell surface moiety is CD137 or another immune effector co-stimulatory moiety and/or wherein at least one cell surface moiety is PD-L1 or another tumor-associated moiety or immune checkpoint molecule.

75. The method according to claim 72, wherein the reference agent is a multispecific antibody that specifically binds to CD137 or another immune effector co-stimulatory moiety and PD-L1 or another tumor-associated moiety or immune checkpoint moiety.

76. A kit of parts comprising at least two binding molecules that specifically bind to a first and second cell surface moiety, optionally wherein one of the binding molecules comprises a molecular tag attached thereto via a cleavable linker and the other binding molecule comprises a cleavage inducing moiety, and instructions to contact a patient sample with the at least two binding molecules, optionally to induce cleavage of the molecular tag; and to detect the presence or absence of a signal induced by contacting the patient sample with the at least two binding molecules.
